(19) United States
(12) Patent Application Publication
(10) Pub. No.: US 2022/0369998 A1
(43) Pub. Date: Nov. 24, 2022

(54) METHODS FOR DIAGNOSING AND TREATING NEURAL DISEASES

(71) Applicant: B. G. NEGEV TECHNOLOGIES AND APPLICATIONS LTD., AT BEN-GURION UNIVERSITY, Beer Sheva (IL)

(72) Inventors: Alon FRIEDMAN, Scotch Village (CA); Dan MILIKOVSKY, Tel Aviv (IL); Ronel VEKSLER, Tel Aviv (IL)

(21) Appl. No.: 17/762,173

(22) PCT Filed: Sep. 17, 2020
(86) PCT No.: PCT/IL2020/051033

§ 371 Date: Mar. 21, 2022

Related U.S. Application Data
(60) Provisional application No. 62/902,574, filed on Sep. 19, 2019.

(51) Int. Cl. A61B 5/00; A61B 5/374

(52) U.S. Cl. CPC A61B 5/4064; A61B 5/374; A61B 5/7275; A61B 5/4076; A61B 5/4839

(57) ABSTRACT

The present invention is directed to a method for determining a paroxysmal slow waves event (PSWE) so as to determine blood-brain barrier dysfunction (BBBD) or increased risk of developing a neurological disease or disorder in a subject.

### CROSS-REFERENCE TO RELATED APPLICATIONS

This application claims the benefit of priority of U.S. Provisional Application No. 62/902,574, titled “METHODS FOR DIAGNOSING AND TREATING NEURAL DISEASES”, filed Sep. 19, 2020, the contents of which are incorporated herein by reference in their entirety.

### FIELD OF THE INVENTION

The invention relates generally to the field of neurological diseases and conditions associated therewith.

### BACKGROUND

Neurodegenerative diseases, e.g., Alzheimer's disease (AD), and Parkinson's disease, prevalence is constantly increasing as the world population ages. It is estimated that in 2050 about 80 million will suffer AD worldwide. Understanding the mechanisms underlying neurodegenerative diseases, such as AD, is crucial for the development of early diagnostics and treatment. In addition, a growing body of evidence shows a significant comorbidity of undiagnosed epilepsy among AD patients.

Blood-brain barrier dysfunction (BBBD) was shown to be a key component in the pathogenesis of epilepsy (AKA epileptogenesis) in many studies with a specific role attributed to the extravasation of serum albumin into the brain neuropil. In animal studies, induction of BBBD induces astro-glial dysfunction, neuroinflammation, alterations in the extracellular matrix, excitatory synaptogenesis, pathological plasticity, and increase in neural excitability, which reduce seizure threshold and precede neurodegeneration.

Gross slowing of electroencephalogram (EEG) activity has been observed in AD and other types of dementia, however, there has not been a detailed investigation of the temporal characteristics of this generalized slowing.

There is still a great need for a biomarker for early diagnosis of neural networks' slowing or low frequency activity.

### SUMMARY

The following embodiments and aspects thereof are described and illustrated in conjunction with systems, tools and methods which are meant to be exemplary and illustrative, not limiting in scope.

According to a first aspect, there is provided a method for determining blood-brain barrier dysfunction (BBBD) in a subject, comprising determining a paroxysmal slow waves event (PSWE) in the subject, wherein the PSWE has a median frequency (MF) of 3-10 Hz and is at least 5 seconds long, thereby determining BBBD in the subject.

According to another aspect, there is provided a method for determining a subject is at increased risk of developing a neurological disease or disorder, comprising determining a PSWE in the subject, wherein the PSWE has a MF of 3-10 Hz and is at least 5 seconds long, thereby determining the subject is at increased risk of developing a neurological disease or disorder.

According to another aspect, there is provided a method for treating a neurological disease or disorder in a subject in need thereof, comprising: (a) determining whether the subject has a PSWE having a MF of 3-10 Hz and being at least 5 seconds long; and (b) administering to the subject determined as having a PSWE having a MF of 3-10 Hz and being at least 5 seconds long, a therapeutically effective amount of a BBB permeability-rectifying agent, thereby treating a neurological disease or disorder in the subject.

In some embodiments, the BBBD comprises increased BBB permeability, compared to a BBB control.

In some embodiments, the PSWE having a MF of 3-10 Hz and being at least 5 seconds long, is indicative of the subject being at increased risk of developing a neurological disease or disorder, compared to a control.

In some embodiments, the determining is by electroencephalogram (EEG).

In some embodiments, the PSWE is determined in the cerebral cortex of the subject.

In some embodiments, the neurological disease or disorder is selected from the group consisting of: Alzheimer's disease, Parkinson's disease, Havana syndrome, and a bipolar disorder.

In some embodiments, the subject is afflicted with a head trauma, BBB dysfunction (BBBD), or both.

In some embodiments, the method further comprises a step of treating the subject with a BBB permeability-rectifying agent.

In some embodiments, the PSWE has a MF of 5 Hz at most.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the figures and by study of the following detailed description.

### BRIEF DESCRIPTION OF THE FIGURES

FIGS. 1A-1I include graphs and images showing that dementia-related slow network activity is rather continuous but composed of distinct paroxysmal slow waves events (PSWE). (1A) Spectral analyses of AD patients (N=16), mild cognitive impairment (MCI) patients (N=12) and age matched controls (N=11). (1B) Quantification by area under the curve of spectral analyses revealed a significantly lower alpha relative power in patients with AD (3) (8-12 Hz, p=0.007), compared to control (1) and MCI patients (2). AD patients had significantly greater relative power in theta band (5-8 Hz, p=0.015) and significantly lower relative power in alpha (p=0.006) compared to age-matched controls (1). (1C) A PSWE detected in an AD patient. Traces from electrodes P3 (upper trace) and P4 (bottom trace, averaged as reference) are shown. The segment within the dashed rectangle in P3 is magnified. The median power frequency is presented below each trace. Segments below 6 Hz (dashed line) are marked by full line. (1D) Gaussian mixture model algorithm classified values of PSWE occurrence per minute from each patient into two groups (p<0.0001). (1E) Patients within the high PSWE rate (H-PSWE) group were significantly more likely to be diagnosed with AD compared with those from the low PSWE rate (L-PSWE) group (p=0.00159). (1F) Patients in the H-PSWE group have significantly lower Mini-Mental State Examination (MMSE) score (p=0.0078) than the L-PSWE group. (1G) MMSE is inversely correlated with PSWE rate (p=0.006). (1H) PSWEs in the H-PSWE group show a significantly longer duration (p<0.0001). (1I) Mean heat maps of PSWE per minute in each electrode. *p<0.05, **p<0.01, ****p<0.0001.

FIGS. 2A-2G include graphs and images showing PSWEs and blood-brain barrier dysfunction (BBBD) in patients with epilepsy. (2A) PSWE occurrence is higher among patients with epilepsy (N=17) compared with aged-matched controls (N=9, p=0.0052). (2B) Prevalence of epilepsy diagnosis is more common in the H-PSWE group compared with the L-PSWE group. Data shown in percentage (p=0.02). (2C) BBBD is significantly greater among epilepsy patients (N=12) compared with aged-matched controls (N=59, p=0.006). (2D) Prevalence of epilepsy diagnosis is significantly more common among high volume of BBBD (H-BBB) group compared with the low LBBBD (L-BBB) group. Data is shown in percentage (p=0.007). (2E) BBBD value by lobe with respect to PSWE occurrence per minute within the same lobe show a significant spearman correlation in a patient with epilepsy (r=0.93, p=0.002). Positive spearman's r values were found in 8 of 10 patients. The correlation was statistically significant for 3 of the patients, all of them with positive spearman values. (2F-2G) A permeability map of BBBD (2F, radiological view) and PSWE per minute heat map (2G, radiological view) of the same patient respectively; see Materials and methods in the Example section). *p<0.05; **p<0.01.

FIGS. 3A-3L include graphs and micrographs showing that slowed network activity among aged animals comprises PSWE. (3A) Following automatic detection of PSWEs in electrocorticography (ECOG) recordings of young (N=5) and old (N=12) mice, were blindly classified by Gaussian Mixture Model into 2 groups according to the PSWE occurrence per minute—high PSWE rate (HPSWE) and low PSWE rate (LPSWE). (3B) Spectral analysis showed significantly (p=0.002) greater delta and significantly (p=0.002) lower alpha relative power (according to area under the curve) in old HPSWE (Old patho) compared with old LPSWE (old). Old LPSWE animals had significantly greater theta relative power compared with young ones (p=0.04, FIG. 3B). (3C) Spectral analysis was re-calculated after PSWEs were excluded from the signal. Differences in theta relative power were diminished. (3D) PSWEs of old pathological (old patho) animals show significantly greater duration (p<0.0001) compared with young. PSWEs of the old group may last up to more than 100 s. (3E) PSWEs of old (H and LPSWE) animals show significantly lower (p<0.0001) median frequency distribution compared with young. (3F) PSWEs are more common (p<0.0001) among old HPSWE animals compared with young or old LPSWE throughout day and night. (3G-3I) Fluorescent staining of brain tissue taken from a young mouse (N=3, 3 months of age, indicative of neurons (NeuN) (3G), glial fibrillary acidic protein (GFAP) (3H), and Albumin (3I). (3J-3L) Staining of tissue taken from an old mouse (N=4, 21 months of age). NeuN (3J), GFAP (3K), and Albumin (3L). In (3G-3L), scale bar=50 μm. *p<0.05; **p<0.01.

FIGS. 4A-4J include graphs and images showing PSWE and BBBD in the status epilepticus model of epilepsy. (4A) BBBD volume is significantly (p=0.001) greater among epileptic rats (N=10) compared with naive control (N=12). (4B-4C) MRI scans of control and epileptic animals, respectively; (see Materials and methods in the Example section). (4D) PSWEs occurrence per minute (averaged in 5 recording days) is greater (p=0.055) among epileptic rats (N=5) compared with naïve (N=4). (4E) PSWEs are significantly longer (p=0.02) in epileptic rats compared with naïve controls. (4F) PSWEs are significantly (p=0.03) slower in epileptic rats compared with naïve controls. (4G-4H) Spectral analysis of ECOG shows significantly greater power (according to area under the curve, AUC) in delta (p=0.03, 1-6 Hz) among epileptic (1) rats compared with naïve (2) and significantly lower relative power in alpha (p=0.03, 8-12 Hz) and beta (p=0.03, 12-20 Hz). (41) Seizures (N=18) recorded from the same epileptic rats are significantly (p=0.0007) shorter compared to PSWEs (N=4885). Plot shows median, min and max values. (4J) Seizures (N=18) are significantly (p<0.0001) faster than PSWEs (N=4885). Plot shows median, min and max values.

FIGS. 5A-5G include micrographs and graphs showing that long-term albumin perfusion may serve as a model of chronic BBB disruption and induces PSWE comprised-network activity. (5A-5B) Nucleic acid (DAPI), GFAP, and albumin-specific fluorescent staining of tissue taken from a rat intraventricularly treated with albumin for 28 days. (5A) Untreated hemisphere. (5B) Treated hemisphere. Scale bar is 50 μm. (5C) Spectral analyses of ECOG recordings reveal slow frequency dominance among long-term albumin-treated animals (N=8) compared with those treated with artificial cerebral spinal fluid (ACSF; N=5), with a greater relative power in the 1-5 Hz band (p=0.002), and lower in the 8-12 Hz (p=0.0.002) and 12-20 Hz bands (p=0.003). (5D) When PSWEs are excluded from the recordings of both groups, the level of significance is 0.0016, 0.0031, 0.0031 for Delta, alpha and beta, respectively. (5E) PSWE are more common in the ipsilateral (“ipsi”) hemisphere compared with the contralateral (“contra”) hemisphere, relative to the point of injection, in albumin-treated (“alb”) animals (p=0.008), and compared to the ipsilateral hemisphere of ACSF-treated (“acsf”) controls (p=0.007) **p<0.01. (5F) PSWEs detected in albumin-treated animals show significantly longer duration compared with those recorded in ACSF-treated controls (p<0.0001). (5G) PSWEs detected in albumin-treated animals show significantly lower median frequency (median frequencies of each PSWE are shown in histogram) compared with the ACSF-treated group (p<0.0001).

FIG. 6 includes a graph showing locomotor activity of old animals during PSWE. The histogram shows mean (dashed line) locomotor activity of 5 old animals (shadow is standard error). Animals spent up to 20% of the PSWE time in a zero-movement status.

FIGS. 7A-7F include graphs and maps showing that extensive BBB leakage is associated with a worse neuropsychiatric status in bipolar patients. (7A) Quantification of the normalized contrast-agent accumulation-rate in each voxel allowed the mapping of BBB permeability. Representative BBB maps of five bipolar patients showcase the different extents of damage among the bipolar cohort (displayed slices were selected to represent maximal BBB leakage in each subject). (7B) The overall percent of brain volume with leaky BBB was quantified in all patients and controls, revealing a high variability of values among the bipolar cohort. (7C) Blinded K-means clustering of all 50 subjects has identified a group with “extensive BBB leakage”, comprising ten bipolar patients, and a group with “normal BBB leakage”, comprising 26 patients and 14 controls (p<0.0001). (7D) The percent of region-specific BBB leakage was calculated within 126 brain regions and compared between the two clusters of bipolar patients. Compared to bipolar patients with normal BBB leakage, the “extensive BBB leakage” group had significantly higher levels of leakage, compared to “normal BBB leakage”, in 112 of the 126 regions (Wilcoxon rank sum test with a false discovery rate correction for multiple comparisons). The 112 significantly different regions are depicted in (7D), with the values corresponding to the average percent of tissue with BBB leakage within each region. (7E) Representative courses of illness show an episodic course in patients with normal BBB leakage (patients I and II), and a worsening course in patients with extensive BBB leakage (patients III and IV). (7F) Quantitative analysis confirmed the higher incidence of a chronic (vs. episodic) course of illness among patients with extensive BBB leakage. Extensive BBB leakage was also associated with a greater severity of depression (Montgomery-Åsberg Depression Rating Scale, MADRS), elevated anxiety (Hamilton Anxiety Rating Scale, HAM-A), and worse social/occupational functioning (Global Assessment of Functioning, GAF). Statistical comparisons were conducted using the Wilcoxon rank sum test. Error bars denote standard error of the mean. Asterisks denote level of significance, with * for p≤0.05, ** for p≤0.01, and *** for p≤0.001.

FIG. 8 includes vertical bar graphs showing that extensive BBB leakage is associated with metabolic dysregulation. Bipolar patients with extensive BBB leakage were found to have higher body mass indices, increased risk of cardiovascular disease, advanced heart age, and higher levels of insulin resistance. Statistical comparisons were conducted using the Wilcoxon rank sum test. Error bars denote standard error of the mean. Asterisks denote p≤0.05. HOMA-IR, homeostatic model assessment of insulin resistance.

FIGS. 9A-9B include graphs showing that progressive BBB dysfunction in aging mice is associated with aberrant network activity. (9A) A representative trace of ECOG recording showing a PSWE with slow wave activity less than 5 Hz within a 10-s window (marked with *). (9B) Number of PSWEs per day was counted and compared between young (n=5) and old mice (n=18) (Mann-Whitney test, P=0.02). For all tests, *P<0.05, **P<0.01, and ****P <0.001.

FIGS. 10A-10C include micrographs and graphs showing that diffusion tensor imaging reveal fiber-specific changes among subjects stationed in Cuba (e.g., exposed individuals). (10A) Streamlines colored by direction that correspond to pixels indicating a significant difference in fiber density between non-exposed (not stationed in Havana, or tested before being stationed in Havana) and exposed (tested within one month after returning from Havana; P<0.05, age and error-corrected). (10B) A decrease in fiber density was observed predominantly along the right crus of the fornix, past the hippocampal commissure and projecting into the hippocampus, as well as in the splenium of the corpus callosum. (10C) Post-hoc analysis showed decreased fiber density in affected regions among exposed individuals, particularly among the remotely exposed group.

FIGS. 11A-11M include graphs and maps showing BBB dysfunction in subjects stationed in Cuba (e.g., following exposure). (11A-11B) Typical scan from an individual both prior (11A) to living in Havana and within 30 days of returning (11B; after a stay of approximately 6 months). Voxels with a leaky BBB (>95th percentile of controls) are highly prominent. (11C) Living in Havana was associated with an increase in the % voxels with BBBD (Wilcoxon, P=0.06). (11D-11E) Regional analysis of 11A and 11B, respectively, showed brain regions with a leaky BBB of >3 times the standard deviation (>3 SD) of controls, mainly in the right hemisphere (11E; pre- and post-exposure). (11F) The number of leaky regions (>3 SD of controls) at pre- and post-exposure (Wilcoxon, P=0.06). (11G-11M) Six (6) (out of 126) brain regions were found to show a statistically significant increase in contrast accumulation (i.e., a leaky BBB) in post- compared to pre-exposure scans (P<0.05). (11H) Left pallium; (11I) Right anterior insula; (11J) Right basal forebrain; (11K) Right posterior orbital gyrus; (11L) Right posterior frontal gyrus; and (11M) Right posterior occipital gyrus.

FIGS. 12A-12G include maps and graphs showing region-specific BBB dysfunction in subjects stationed in Cuba (e.g., following exposure). (12A-12B) Comparison of averaged z-score for BBB integrity between non-exposed (12A) and exposed groups (12B). (12C) Comparison of overall percentage of brain volume with a leaky BBB between non-exposed and exposed groups. (12D) Number of regions with a leaky BBB, of >3 times the standard deviation (>3 SD) of controls, in exposed compared to non-exposed groups. (12E) Number of regions with BBB leakage (>3 SD of controls) as a function of time since last exposure (Zero-inflated quasi-Poisson regression showing non-significant negative relationship, β=−0.051, p=0.16, ϕ=22.4). (12F-12G) Among the pre- and post-exposure (i.e., double-tested) group, regions discovered to be significantly leaky only following exposure (N=6, FIGS. 11G-11M) were next examined in the broader group. Shown here is a left (12F) and right (12G) comparison of these regions between exposed (N=17) and non-exposed (N=18) subjects. The inventors also tested the hypothesis that BBB integrity would be impaired in the parahippocampus gyrus due to its well-recognized role in spatial memory (see text). Regions: Basal forebrain (B Foreb), Anterior Cingulate (Ant Cing), Superior Orbital (Sup Orb), Superior Frontal (Sup Front), Superior occipital (Sup Occip) and parahippocampal (Parahipp) gyri. Error bars represent standard error of the mean (SEM).

FIGS. 13A-13I include maps and graphs showing that magnetoencephalography (MEG) reveals paroxysmal slowing of brain activity in subjects stationed in Cuba. (13A-13B) Spectrogram (eyes closed) from the same individual pre- (13A; left plates) and post-exposure (13B; right plates). Note the clear dominant 10 Hz rhythm prior to posting and intermittent slowing following. (13C) Group comparison of spectral analysis (eyes closed). (13D) Sum power for each frequency band. Note the reduction in alpha band power and increase in delta among the exposed group. (13E) Slowing of activity was composed of paroxysmal slow wave events (PSWEs). The upper trace is the original MEG recording, and the calculated median frequency below. A PSWE was defined as the time window in which the median frequency was <6 Hz for >5 sec. (13F-13G) PSWE frequency and spatial occurrence (% of MEG channels with independent PSWEs) in exposed compared to non-exposed groups. (13H) PSWE frequency with time lapsed from last exposure. Tweedie GLM regression line shows a significant negative relationship (β=−0.088, p=0.022*, ϕ=0.623). (13I) Brain surface showing spatial distribution of PSWEs. Non-exposed controls (Non): N=56; recent, N=11; remote, N=10. Error bars represent SEM.

FIGS. 14A-14I include images and graphs showing fumigation in Havana and cholinesterase inhibitor exposure. (14A) Outdoor and (14B) indoor fumigation in Cuba for the eradication of mosquitos as initiated by the Cuban government. (14C) Number of signed annual outdoor fumigations in Canadian diplomatic residences between 2016-2018. Note the sharp increase in frequency during 2017 prior to the appearance of symptoms (mean and range). (14D-14E) Group comparison did not show significant differences in serum activity for either butyryl- (14D) or acetyl- (14E) cholinesterase (non-exposed (Non): N=12; exposed: N=20). (14F) Butyryl- and (14G) acetyl-cholinesterase activity is increased with time since exposure. (14H) Serum butyryl- and (14I) acetyl-cholinesterase activity in recently (N=11) and remotely (N=9) exposed individuals. Error bars represent SEM.

### DETAILED DESCRIPTION

In some embodiments, the present invention is directed to a method for determining blood-brain barrier dysfunction (BBBD), or a condition or a disease associated therewith, wherein the method comprises determining a paroxysmal slow waves event (PSWE) in the subject.

According to some embodiments, a method for determining blood-brain barrier dysfunction (BBBD) in a subject, comprising determining a paroxysmal slow waves event (PSWE) in the subject, thereby determining BBBD in the subject, is provided.

According to some embodiments, a method for determining a subject is at increased risk of developing a neurological disease or disorder, comprising determining a PSWE in the subject, thereby determining the subject is at increased risk of developing a neurological disease, is provided.

In some embodiments, the term “BBBD” encompasses any functional damage, structural damage, or both, to the BBB. In some embodiments, the BBBD comprises micro-vasculopathy, extravasation of a serum protein, e.g., albumin, or a combination thereof.

In some embodiments, BBBD comprises increased BBB permeability, compared to a BBB control.

In some embodiments, a BBB control is an intact BBB. In some embodiments, a BBB control is a healthy BBB. In some embodiments, a BBB control is a BBB of a healthy subject. In some embodiments, a BBB control is devoid of excessive permeability. In some embodiments, a BBB control is devoid of micro-vasculopathy. In some embodiments, a BBB control is devoid of extravasation of a serum protein, e.g., albumin.

As used herein, the term “increased BBB permeability” refers to a pathological state or an abnormal state of uncontrollable or unregulated leakage of blood components from a blood vessel to a brain tissue.

Other than the method disclosed herein, BBB permeability can be determined by MRI, such as dynamic contrast enhanced (DCE)-MRI, which requires administration of a contrasting agent, as would be apparent to one of ordinary skill in the art.

In some embodiments, increased is by at least 5%, at least 10%, at least 20%, at least 35%, at least 50%, at least 75%, at least 100%, at least 200%, at least 350%, at least 400%, at least 500%, at least 750%, or at least 1,000% more than control, or any value and range therebetween. Each possibility represents a separate embodiment of the invention. In some embodiments, increased is by 1-20%, 10-50%, 30-90%, 80-150%, 100-250%, 200-500%, 350-750%, or 650-1,000% compared to control. Each possibility represents a separate embodiment of the invention.

As used herein, the term “PSWE” refers to transient paroxysmal slowing of the cortical network, which reflects cortical slowing.

In some embodiments, the PSWE is recorded. In some embodiments, PSWE is recorded by MEG. In some embodiments, the PSWE is recorded. In some embodiments, PSWE is recorded by EEG. In some embodiments, PSWE is recorded by intracortical recording. In some embodiments, PSWE is recorded by ECoG.

In some embodiments, methods for recording PSWE are common and would be apparent to one of ordinary skill in the art. In some embodiments, PSWE is recorded by any method known to one of skill in the art to be suitable for recording PSWE, as described herein.

In some embodiments, a PSWE has a median frequency (MF) of 2-10 Hz, 5-9 Hz, 1-5 Hz, 5-8 Hz, 5-7 Hz, 5-6 Hz, 6-9 Hz, 3-9 Hz, 4-8 Hz, or 6-7 Hz. Each possibility represents a separate embodiment of the invention. In some embodiments, a PSWE has a MPF of 9 Hz at most, 8 Hz at most, 7 Hz at most, 6 Hz at most, 5 Hz at most, 4 Hz at most, 3 Hz at most, 2 Hz at most, or 1 Hz at most, or any value and range therebetween. Each possibility represents a separate embodiment of the invention.

As used herein, the terms “median power frequency (MPF)” and “median frequency (MF)”, are interchangeable.

In some embodiments, MF comprises MPF.

In some embodiments, the PSWE is at least 1 second long, at least 2 seconds long, at least 3 seconds long, at least 4 seconds long, at least 5 seconds long, at least 7 seconds long, at least 10 seconds long, at least 20 seconds long, at least 30 seconds long, or at least 60 seconds long, or any value and range therebetween. Each possibility represents a separate embodiment of the invention. In some embodiments, the PSWE is 1-20 seconds long, 5-10 seconds long, 4-30 seconds long, 5-25 seconds long, 2-13 seconds long, 10-45 seconds long, or 5-60 seconds long. Each possibility represents a separate embodiment of the invention.

In some embodiments, the PSWE has a MPF of 2-10 Hz, 5-9 Hz, 1-5 Hz, 5-8 Hz, 5-7 Hz, 5-6 Hz, 6-9 Hz, 3-9 Hz, 4-8 Hz, or 6-7 Hz, and is 1-20 seconds long, 5-10 seconds long, 4-30 seconds long, 5-25 seconds long, 2-13 seconds long, 10-45 seconds long, 5-60 seconds long. In some embodiments, the PSWE has a MPF of 9 Hz at most, 8 Hz at most, 7 Hz at most, 6 Hz at most, 5 Hz at most, 4 Hz at most, 3 Hz at most, 2 Hz at most, or 1 Hz at most, and is at least 1 second long, at least 2 seconds long, at least 3 seconds long, at least 4 seconds long, at least 5 seconds long, at least 7 seconds long, at least 10 seconds long, at least 20 seconds long, at least 30 seconds long, or at least 60 seconds long, or any value and range therebetween. Each possibility represents a separate embodiment of the invention.

In some embodiments, a PSWE having a MPF of 3-10 Hz and being at least 5 seconds long, is indicative of the subject being at increased risk of developing a neurological disease or disorder, compared to a control subject.

In some embodiments, a PSWE having a MPF of 3-10 Hz has a MPF of 5 Hz at most.

In some embodiments, a frequency of at least 1 PSWE per minute, at least 2 PSWE per, or at least 3 PSWE per minute, or any value and range therebetween, is indicative of the subject being at increased risk of having increased risk or probability of developing a neurological disease or disorder. Each possibility represents a separate embodiment of the invention. In some embodiments, a frequency of 1-3 PSWE per minute, 1-2 PSWE per minute, or 2-3 PSWE per minute, is indicative of the subject being at increased risk of developing a neurological disease or disorder. In some embodiments, increased risk or probability is compared to a control.

In some embodiments, a control subject has an intact BBB. In some embodiments, a control subject is a healthy subject. In some embodiments, a control subject is devoid of excessive BBB permeability. In some embodiments, a control subject is devoid of brain micro-vasculopathy. In some embodiments, a control subject is devoid of neural extravasation of a serum protein, e.g., albumin.

Methods for determining BBB intactness, permeability, and micro-vasculopathy are common and would be apparent to one of ordinary skill in the art of neurology. Non-limiting examples for such methods include, but are not limited to MRI, such as DCE-MRI, as exemplified herein below.

In some embodiments, the method comprises determining PSWE by electroencephalogram (EEG).

As used herein, the term “EEG” refers to any electrophysiological monitoring method which records the brain's electrical activity, including MEG. In some embodiments, the monitoring comprises monitoring the electrical activity, magnetic activity, or both, of the brain.

In some embodiments, the method comprises determining the PSWE in or out of the cerebral cortex of the subject. In some embodiments, the PSWE is determined in the cerebral cortex of the subject.

Methods for monitoring the brain's electrophysiological activity, in general, and the cerebral cortex's electrophysiological activity, in particular, such as by means of EEG or MEG, would be apparent to one of ordinary skill in the art. A non-limiting exemplary EEG process for the aforementioned measuring, is delineated hereinbelow (see Example section).

As used herein, “neurological disease” refers to any disorder related to a component of the neural system, e.g., brain, spinal cord, or other nerves. In one embodiment, neurological disease includes but is not limited to biochemical, electrical, or structural abnormalities in components of the neural system, including neuronal cells, blood vessels of the neural system, or a combination thereof.

According to some embodiments, symptoms of a neurological disease or disorder are selected from: loss of short-term memory (e.g., asking repetitive questions, frequently misplacing objects or forgetting appointments), cognitive deficits (e.g. impaired reasoning, difficulty handling complex tasks, and poor judgment), language dysfunction (aphasia, e.g., difficulty thinking of common words, errors in speaking and/or writing), visuospatial dysfunction (agnosia, e.g., inability to recognize faces or common objects), resting tremor, rigidity, slow movements, postural instability, apraxia, dementia, sleep disorders, depression, apathy, irritability, anhedonia, antisocial behavior, full-blown bipolar or schizophreniform disorder, chorea, myoclonic jerks, and pseudo-tics, a puppet-like gait, facial grimacing, inability to intentionally move the eyes quickly without blinking or oculomotor apraxia, inability to sustain a motor act, olivopontocerebellar atrophy, ataxia, dysmetria, dysdiadochokinesia, poor coordination, orthostatic hypotension, urinary retention or incontinence, constipation, erectile dysfunction, decreased sweating, difficulty breathing and swallowing, fecal incontinence, decreased tearing and salivation, REM sleep behavior disorder (e.g., speech or skeletal muscle movement during REM sleep), respiratory stridor, a seizure, epilepsy, an infection (such as in the CNS), a metabolic disorder, exposure to a toxin (such as a neurotoxin), concussion, and trauma (such as brain injury).

In some embodiments, neurological diseases and disorder comprise neurodegenerative disease, and neuromuscular diseases, selected from: autonomic neuropathies, Horner syndrome, multiple system atrophy, pure autonomic failure, delirium, dementia, Alzheimer's disease, chronic traumatic encephalopathy, frontotemporal dementia, Lewy body dementia, Parkinson's disease, multiple sclerosis, neuromyelitis optica, Huntington's disease, progressive supranuclear palsy, neuro-ophthalomologic and cranial nerve disorder, Isaacs Syndrome, Stiff-Person syndrome, Guillain-Barré syndrome (GBS), chronic inflammatory demyelinating polyneuropathy (CIDP), hereditary neuropathies, hereditary motor neuropathy with liability to pressure palsies (HNPP), amyotrophic lateral sclerosis (ALS) and other motor neuron diseases (MNDs), myasthenia gravis, nerve root disorders, herniated nucleus pulposus, peripheral neuropathy, mononeuropathies, multiple mononeuropathy, polyneuropathy, brachial plexus and lumbosacral plexus disorders, spinal muscular atrophies (SMAs), thoracic outlet compression syndromes (TOS), Creutzfeldt-Jakob Disease (CJD), Gerstmann-Sträussler-Scheinker Disease (GSS), seizure disorders, spinal cord disorders and stroke.

In some embodiments, a neurological disease or disorder is Havana syndrome. As used herein, the term “Havana syndrome” refers to a variety of health problems reported by foreign embassy staff members (e.g., US, Canada) situated in Cuba.

In some embodiments, a neurological disease or disorder comprises a brain injury. In some embodiments, a brain injury originates from an unknown source. In some embodiments, a brain injury is toxin-induced or toxin-related brain injury. In some embodiments, a brain injury is metabolic-induced or metabolic-related brain injury. In some embodiments, a brain injury is induced by or relates to an infectious agent or infection. In some embodiments, the brain injury is a result of an infection. In some embodiments, the brain injury is characterized, manifests, observable, determinable, detectable, or any combination thereof, after an infection event, e.g., post-infection.

In some embodiments, a neurological disease or disorder is a bipolar disorder. As used herein, the term “bipolar disorder” encompasses any disorder characterized by episodes of mania and depression, which may alternate, although a predominance of one or the other, was reported in many subjects.

In some embodiments, a neurological disease or disorder is selected from: Alzheimer's disease, Parkinson's disease, Havana syndrome, and a bipolar disorder.

The term “subject” as used herein refers to an animal, including a non-human mammal, and human organism. In one embodiment, a subject in need thereof is a subject afflicted with and/or at risk of being afflicted with a condition associated with neurological disease or disorder. In one embodiment, a subject in need thereof is a subject afflicted with and/or at risk of being afflicted with a condition associated with increased BBB permeability.

In some embodiments, the subject is afflicted with a head trauma, BBB dysfunction (BBBD), or both.

In some embodiments, the method of further comprises a step of treating the subject with a BBB permeability-rectifying agent.

As used herein, the term “BBB permeability-rectifying agent” refers to any agent capable of: restoring BBB permeability levels to a normal or healthy level, reduce BBB permeability, increase BBB impermeability, reduce BBB leakage, maintain BBB intactness, or any combination thereof.

In some embodiments, the agent is a small molecule. In some embodiments, the agent is a peptide. In some embodiments, the agent is a nucleic acid. In some embodiments, the agent is an organic or inorganic compound. In some embodiments, the agent is non-invasive brain stimulation (NIBS) therapy.

As used herein, the term “NIBS” refers to any stimulation technique aiming to alter brain activity, including BBB permeability, BBB functionality, or any combination thereof, by induction of an electrical, and/or magnetic stimulation of the brain.

Types and procedures for applying NIBS would be apparent to one of ordinary skill in the art of neurotherapy. Non-limiting examples of NIBS include, but are not limited to, repetitive transcranial magnetic stimulation (rTMS), deep TMS, cranial electrotherapy stimulation (CES), transcranial direct current stimulation (tDCS), transcranial random noise stimulation (tRNS), reduced impedance non-invasive cortical electrostimulation (RINCE), and electroconvulsive therapy (ECT).

According to some embodiments, a method for treating a neurological disease or disorder in a subject in need thereof, comprising: (a) determining whether the subject has a PSWE having a MF of 3-10 Hz and being at least 5 seconds long; and (b) administering to the subject determined as having a PSWE having a MPF of 3-10 Hz and being at least 5 seconds long, a therapeutically effective amount of a BBB permeability-rectifying agent, thereby treating a neurological disease or disorder in the subject, is provided.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or formulations prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the disease severity, delaying the disease onset, reducing the disease cumulative incidence, or any combination thereof.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Material and Methods

### EEG Recordings from AD and MCI Patients

Routine electroencephalogram (EEG) of patients referred to a memory clinic and a control group were retrospectively analyzed. Recordings for 30 minutes were performed using the Nihon Kohden (Japan) Neurofax-1200 with a 32-channel recording in an awake state with open and closed eyes as well as photo-stimulation and a phase of hyperventilation. All EEGs were performed at the Rabin Medical Center, Petach Tikva, Israel. Subjects with mild cognitive impairment (MCI) and dementia were recruited and classified in the Dementia Clinic at Rabin Medical Center (A.G.). In the MCI group, only those patients meeting the criteria for MCI according to the updated guidelines from the NIA and Alzheimer's Association workgroup were included. In brief, the diagnosis MCI requires that (1) disease onset is insidious, (2) there is impairment in one or more cognitive domains without an overt functional impairment and (3) the subject does not meet dementia criteria. Patients classified as suffering from Alzheimer's disease (AD) were included if possible or probable, sporadic AD was present in accordance with the 2011 NIAA guidelines.

### EEG Recordings from Epilepsy Patients

EEG was recorded from epilepsy patients in three medical centers: Rabin Medical Center (N=7 patients, N=9 age-matched controls, 32-channels Nihon Kohden (Japan) Neurofax-1200 system, sampling rate 256 Hz), Wolfson Medical Center, Holon, Israel (N=6 patients, 32-channels, Micromed, Treviso, Italy, sampling rate 256 Hz), and Soroka Medical Center, Beer-Sheva, Israel (N=4, 61 electrodes cap with Ag/AgCl ring electrodes, Micromed, Treviso, Italy, sampling rate 1,024 Hz). EEG was recorded for 20-30 minutes in an awake state with open and closed eyes as well.

### Telemetric ECOG Recording in Rodents

All animal experiments were conducted following an ethical committee approval of the institution in which they were performed, Ben Gurion University of the Negev, Beer-Sheva, Israel or The University of California, Berkeley, Berkeley, Calif., US. Electrodes and wireless transmitters were implanted in the following animal groups: 1) Twelve weeks old (n=5) mice; 2) 18-22 months old (n=12) mice; 3) Nine to 11-weeks-old Wistar male rats implanted with 28 days long intra-cerebroventricular (ICV) perfusion osmotic pumps (N=8, 0.8 mM albumin infusion, N=5 ACSF infusion); 4) Sprague-Dawley rats (n=5, weighing 300 gr) 3 weeks after status epilepticus (SE, see below) and naïve rats (N=3). Groups 1 and 2 electrode coordinates: 0.5 mm rostral or 3.5 mm caudal and 1 mm lateral to bregma, on each side (4 screws, 2 in each hemisphere); Group 3 coordinates: 4.8 mm posterior or 2.7 mm anterior, and 2.2 mm lateral to bregma, on each side (4 screws, 2 in each hemisphere); Group 4 coordinates: 3 mm caudal and 2.5 mm lateral to bregma, on each side (2 screws, 1 in each hemisphere)). Continuous ECoG (sampling rate of 500 Hz) was recorded wirelessly from freely moving animals in their home cage for the described duration of experiments.

### ECoG/EEG Analysis

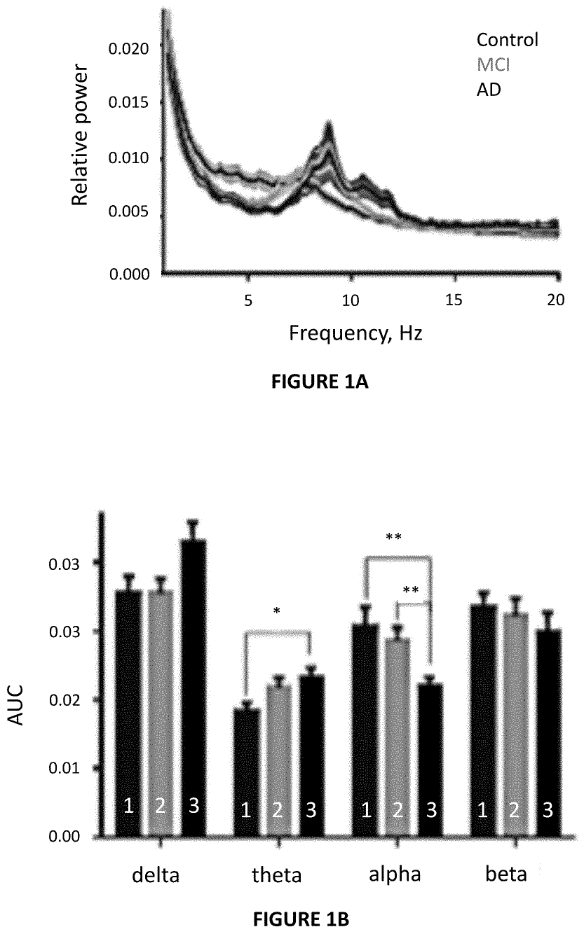

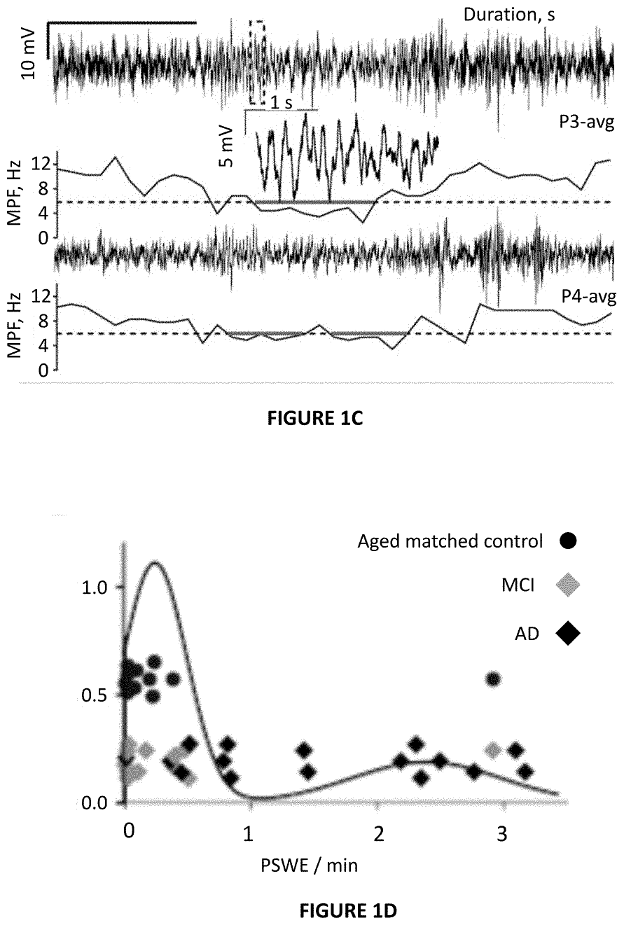

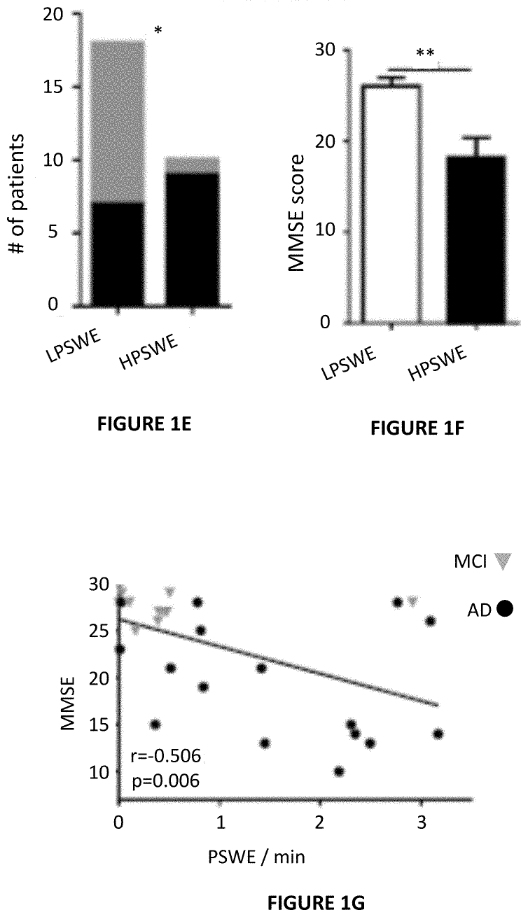

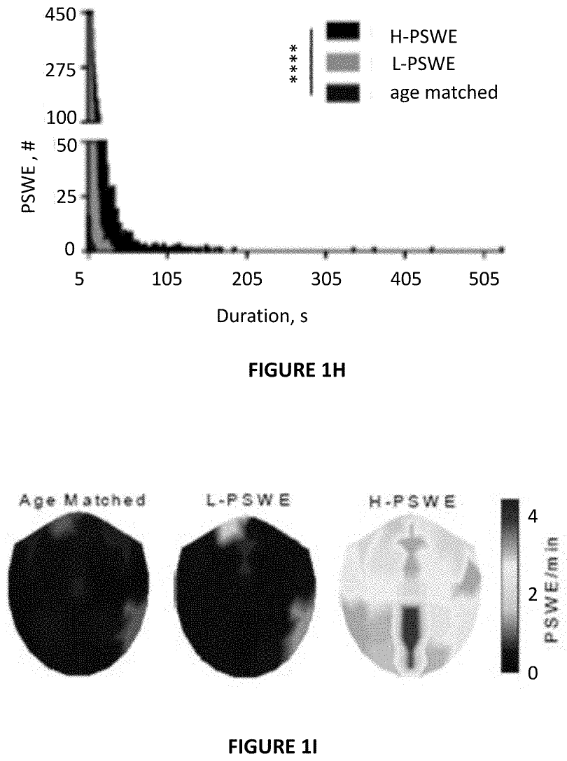

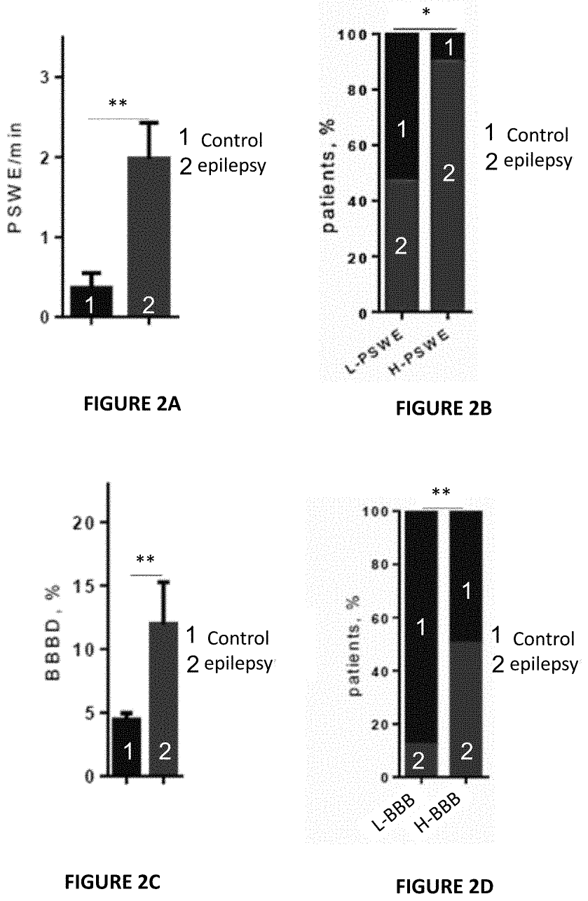

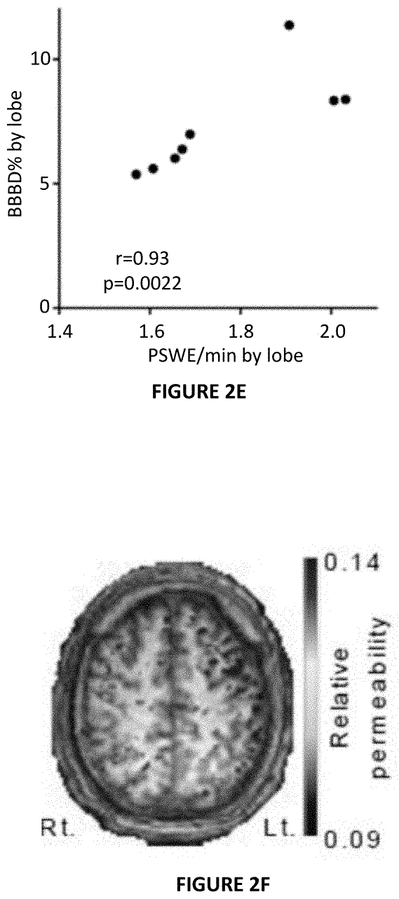

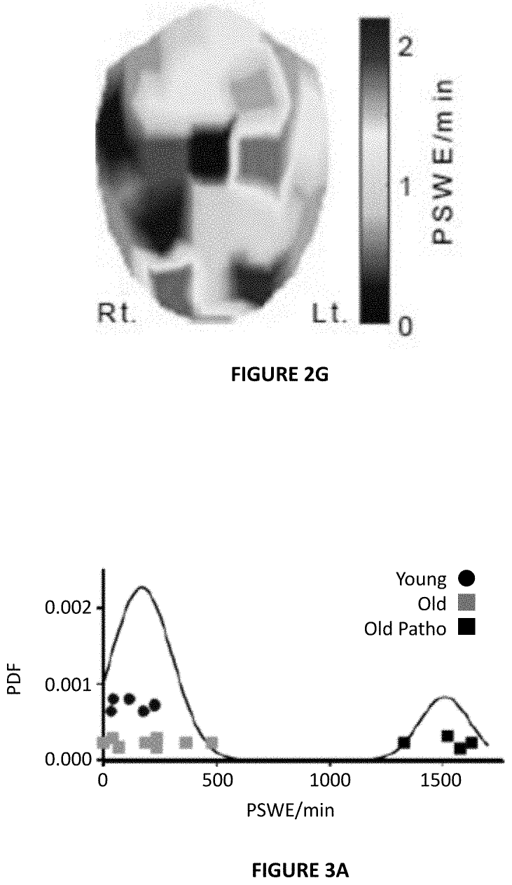

Signal processing was performed offline. Pre-processing included high pass filter (1 Hz), low pass filter (100 Hz) and notch (band-stop) filter (45-55 Hz). EEG human recordings were analyzed as reference to average. Human data were preprocessed by EEGLAB. Animal data were preprocessed using home-developed MATLAB (MathWorks Ltd., MA USA) scripts. To detect PSWE, ECoG or EEG signals were buffered into 2 sec long epochs with 1 sec overlap. Spectral analysis by Fast Fourier Transform (FFT) was applied and the median power frequency (MPF) was extracted for each epoch. PSWE was defined if MPF was <6 Hz for >5 consecutive seconds (see results). Analysis was performed separately for each recorded channel. Power spectrum as in FIG. 1A was calculated for the average channel and for animals ECoG data (e.g., FIG. 2A) over a single channel. FFT was also applied for the entire recording period buffered into 8 seconds epoch with 4 seconds overlap to analyze relative power across the frequency spectrum of 1-20 Hz. Processing was performed by self-developed MATLAB scripts.

### Locomotor Activity

Locomotor activity quantification in freely moving animals was performed using standard methods. Briefly, Signal strength (250 Hz sampling rate) derivative received from the implanted transmitter is correlated with the animal's movement. Data were buffered into 2 sec long epochs with 1 second overlap and mean value was calculated per epoch. Thus, locomotor activity indication was obtained per every second of the recorded period.

### Immunohistochemistry

Postmortem hippocampus was obtained from an AD patient (female, 77 years of age). All participants gave written and informed consent, and all procedures were conducted in accordance with the Declaration of Helsinki and approved by the ethics committee in the University of Bonn. After resection, hippocampi were fixed in 4% formalin and processed into liquid paraffin. All specimens were sliced at 4 μm with a microtome (Microm, Heidelberg, Germany), mounted on slides, dried, and deparaffined in descending alcohol concentration. For mouse samples, mice were anesthetized with Euthasol euthanasia solution and transcardially perfused with ice cold heparinized physiological saline (10 units heparin/mL physiological saline) followed by 4% paraformaldehyde (PFA, Fisher Scientific #AC416785000) in 0.1 M phosphate buffered saline (PBS). Brains were removed, post-fixed in 4% PFA for 24 hours at 4° C., and cryoprotected in 30% sucrose in PBS. Brains were then embedded in Tissue-Tek O.C.T. compound (Sakura, Torrance, Calif.), frozen, and sliced on a cryostat into 20 μm coronal sections, mounted on slides. Samples were stained according to the following protocol. Slides were treated for antigen retrieval (for human, 5 min incubation at 100° C. in sodium citrate buffer, pH 6.0); for mouse, 15 min incubation at 65° C. in Tris-EDTA buffer (10 mM Tris Base, 1 mM EDTA solution, 0.05% Tween 20, pH 9.0), then incubated in blocking solution (5% Normal Donkey Serum in 0.1% Triton X-100/TBS) for 1 hour at room temperature. Samples were then stained with primary antibody at 4° C., followed by fluorescent-conjugated secondary antibody for 1 hour at room temperature, and then incubated with DAPI (900 nM; Sigma-Aldrich) to label nuclei. For human, primary antibodies were rabbit anti-phosphorylated Smad2 (Millipore AB3849-I, 1:500), chicken anti-Albumin (Abcam ab106582, 1:500), and mouse anti-GFAP (Millipore MAB3402, 1:500); for mouse, the same were used except goat anti-GFAP (Abcam ab53554, 1:1000). Secondary antibodies were anti-rabbit Alexa Fluor 568, anti-chicken Alexa Fluor 647, anti-goat Alexa Fluor 488, anti-mouse Alexa Fluor 488 (1:500, Jackson ImmunoResearch), and anti-goat Alexa Fluor 647 (Abcam ab150131, 1:500). All antibodies dilutions were in blocking solution. For tissue from old human patients and aged mice, slide-mounted brain sections for treated with TrueBlack Lipofuscin Autofluorescence Quencher (Biotium #23007) before coverslip mounting.

### Epilepsy Induction by Status Epilepticus in Rats

Status epilepticus was induced in adult Sprague-Dawley rats using paraoxon (intramuscular injection-IM of 0.45 mg/kg, equivalent to 1.4 LD50). Paraoxon injection was followed one minute later by atropine (IM, 3 mg/kg) and toxogonin (IM, 20 mg/kg), to reduce peripheral effects of paraoxon and to lower mortality rate. Rats were monitored for mortality and sickness during the next 48 hours.

### Animal MM BBB Imaging

Animals (N=12 naïve and N=10 epileptic Sprague-Dawley rats) were scanned 1 month after induction of SE (for epileptic group) using the Aspect M2 system (Aspect Imaging Technologies). Briefly, post-contrast signal intensity changes were measured in the whole brain. To measure leakage of the contrast agent, a linear curve was fitted to the dynamic scan intensities of the six consecutive post-contrast T1 -weighted scans (DCE-MRI). That is, a signal s(t) is fitted such that: s(t)=A×t+B, where the slope (A) is the rate of wash-in or wash-out of the contrast agent from the brain parenchyma. BBB score represents the percentage of brain voxels that have a slope value of A>0. A threshold score was defined as the mean +standard deviation of the BBB scores of the control group (12 rats) and BBBD was defined as a score higher than the threshold.

Imaging protocol was approved by the institutional review board of Soroka University Medical Center. All participants signed an informed consent form prior to undergoing the scan. BBB status was measured using DCE-MRI in 125 non-epileptic individuals (age range of 20 to 85 years). BBB permeability was calculated in each brain voxel using an in-house MATLAB script (Mathworks, MA USA). Briefly, a linear regression is applied to the later part of the concentration curve of each voxel (6-20 min); the derived slope is divided by the slope of the superior sagittal sinus, to compensate for physiological (e.g., heart rate, blood flow) and technical (e.g., contrast agent injection rate) variability.

### Participants

A total of 36 adult patients (over 18 years of age) were recruited to the study through the Mood Disorders Clinic (Nova Scotia Health Authority, Canada). Subjects underwent a detailed psychiatric interview using the schedule for affective disorders and schizophrenia (SADS-L) to diagnose bipolar disorder. Diagnoses required a consensus of at least 3 psychiatrists and were based on the DSM-5 criteria. As patients with type I versus II bipolar disorder differ primarily in severity of manic episodes, we did not exclude patients based on this criterion. Mood symptoms were rated using the Montgomery-Á̊sberg depression rating scale (MADRS), Hamilton anxiety rating scale (HAM-A), and the global assessment of functioning scale (GAF, reflecting illness effects on social, occupational, and psychological functioning). Course of illness was determined using the affective morbidity index (AMI, rating the severity and length of manic/depressive episodes), patient interviews, detailed review of medical records, and retrospective analysis of daily mood ratings. Additional data collection included: blood pressure (BP), body mass index (BMI), the homeostatic model assessment of insulin resistance (HOMA-IR, calculated based on fasting levels of blood glucose and insulin), and Framingham risk scores (heart age and risk of cardiovascular disease).

A group of 14 control subjects was also recruited and was matched for sex, age, and metabolic status to the bipolar cohort. The same schedule used for diagnosing bipolar disorder (SADS-L), was used to confirm a lack of psychiatric history in this group. The control group also underwent the above-mentioned protocol of interviews and assessments.

Participants with diabetes, pregnancy, or contradiction to MRI or contrast-enhancement (estimated glomerular filtration rate <60) were excluded from the study. All participants provided informed consent prior to enrollment. The study was approved by the Nova Scotia Health Authority Research Ethics Board (1021507) and adheres to the STROBE statement.

### BBB Imaging

### Acquisition Protocol

Participants were intravenously injected with the magnetic contrast-agent gadoteridol (0.1 mmol/kg, ProHance, Bracco Imaging Canada, Montreal, QC), and its dynamics in the brain were monitored for a period of 20 minutes using T1-weighted MRI (GE Discovery MR750, 3T, FOV 24 cm, slice thickness 6 mm, 192×192 matrix, flip angle 15°, TR/TE 4.1/2.1 ms).

### Image Analysis

Analysis of BBB integrity was performed as published. Briefly, pre-processing included image registration and normalization to MNI coordinates using SPM12 (University College London, www.fil.ion.ucl.ac.uk/spm). The accumulation rate of the contrast-agent during the slow enhancement period of the scan (6-20 min) was calculated for each voxel. To compensate for physiological (e.g., heart rate, blood flow) and technical (e.g., injection rate) variabilities between scans, each voxel's accumulation rate was normalized to that of the superior sagittal sinus. The normalized contrast-agent accumulation rates were defined as the unit-of-measure for BBB permeability, with near-zero/negative values reflecting BBB-protected tissue and positive values representing tissue with tracer accumulation due to cross BBB extravasation. Abnormally high BBB permeability was defined using an intensity threshold of the 95^{th} percentile of all values in a cohort of control subjects. Voxels with values exceeding the intensity threshold (0.02) were considered as tissue with BBB leakage. The percentage of voxels with suprathreshold values was defined as the global measure of BBB dysfunction. To quantify region-specific BBB leakage, each scan was segmented into 126 anatomically/functionally significant areas in accordance with the MNI brain atlas (https://github.com/neurodebian/spm12/tree/master/tpm). The number of voxels with abnormally high BBB permeability (contrast accumulation rates exceeding the above-mentioned intensity threshold) was quantified within each region and divided by the total of voxels comprising the region. This ratio was defined as the percent of region volume affected by abnormally high BBB permeability, and was used as the measure of region-specific BBB leakage.

### Cluster Analysis

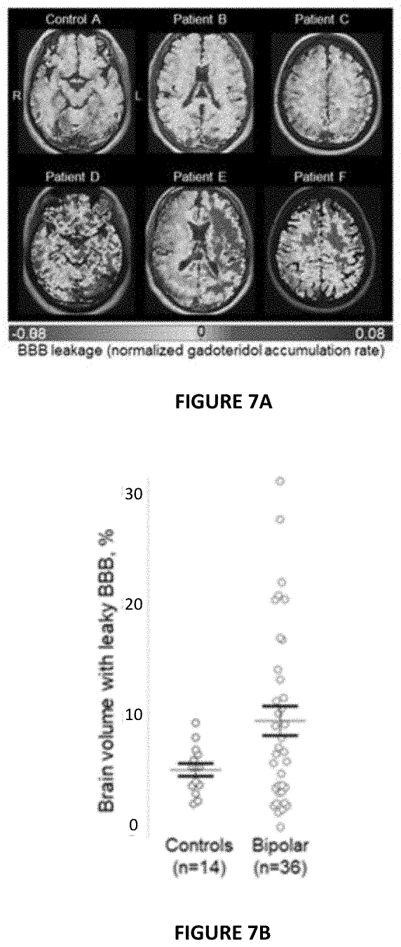

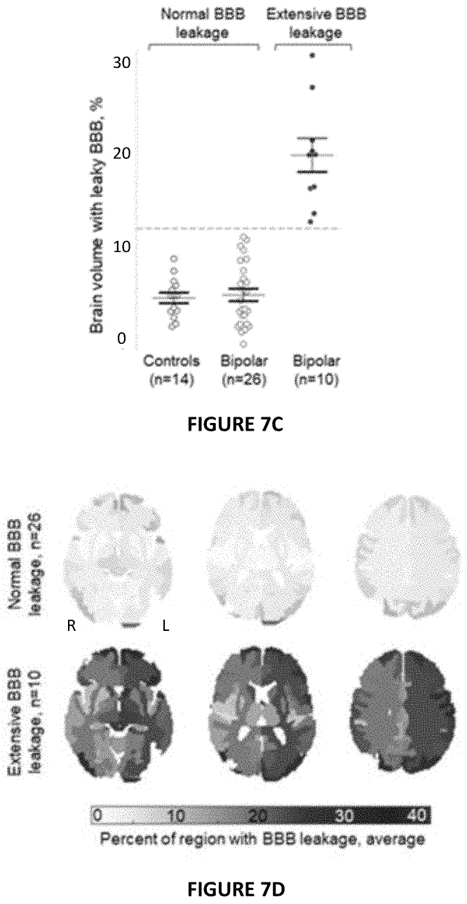

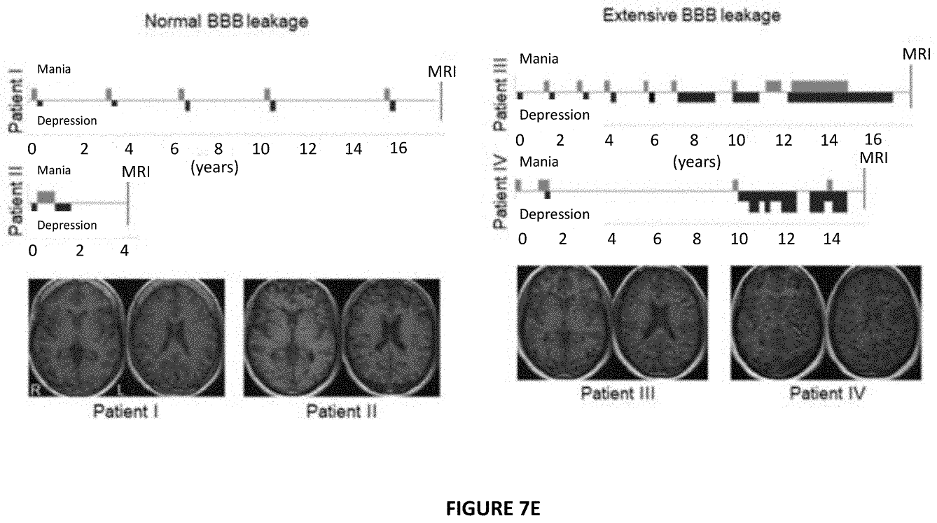

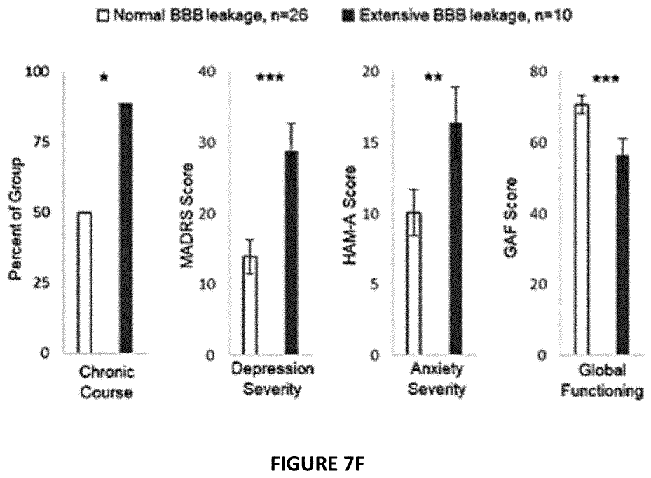

Blinded K-means analysis was used to cluster all subjects based on values of BBB dysfunction (FIG. 7C).

### Statistics

Statistic tests were performed by Prism (GraphPad, Ca, US) unless mentioned otherwise. Mann-Whitney U test was used to compare PSWEs occurrences per minute between groups, relative power frequency bands, PSWE durations and median frequency of PSWEs. Comparing prevalence of AD or epilepsy between groups was done by χ² test. MMSE correlation with PSWE was tested by Pearson's correlation coefficient. PSWE correlation with BBBD was performed by Spearman coefficient correlation. Gaussian Mixture Model for blind clustering was performed by MATLAB.

Continuous variables were compared using the Wilcoxon rank sum test, and categorical variables were compared using either Fisher's or chi test.

### Materials and Methods for Havana Syndrome Study

The study was approved by the Research Ethics Board of the Nova Scotia Health Authority. All participants provided written informed consent.

### Exposed & Non-Exposed Subjects

Between Aug. 10, 2018, and Feb. 20, 2019, the inventors tested 27 Canadian adult subjects referred by Global Affairs Canada for evaluation. Of this group, 23 had been stationed in Havana while the remaining 4 had not. Of those stationed in Havana, 8 were also tested prior to departure (i.e., had repeat testing), providing us with 12 “non-exposed” data sets in total.

### Non-Exposed Controls

In addition to the core group, data from healthy age- and sex-matched individuals outside our cohort was used for the analysis of cognitive and brain imaging data.

### Recent & Remote Exposure

To better understand the progression of injury over time, data sets from the herein described exposed group (N=23) were separated according to when the inventors were able to test the subjects. Specifically, the inventors were able to test 11 individuals within one month of their return from Havana, a group that was classified as “recently exposed,” and 12 individuals 1-19 months after returning (median 14 months), a group the was classified as “remotely exposed.” All 8 individuals tested both pre- and post-exposure were in the “recently exposed” group.

### Duration of Exposure

The duration of stay in Havana ranged from 5 to 8 months (mean 6.5 months) for recently exposed subjects, and from 1 to 48 months (mean 21.95 months) for remotely exposed subjects.

### Assessments

For all 27 subjects, the inventors conducted an initial screening across six dimensions: medical history, self-reported symptom questionnaires, anthropometric measures, computerized cognitive assessments, blood tests, and brain imaging (magnetic resonance imaging (MRI) and magnetoencephalography (MEG)). Exposed individuals suspected to have incurred brain injuries (based primarily on self-reported symptom questionnaires) underwent further neurological, visual, and audiovestibular assessments, as needed.

### Initial Screening

Medical history and anthropometric measures included travel history, height, weight (to calculate BMI), blood pressure, and heart rate.

Self-rated questionnaires included the Rivermead Post-Concussion Symptoms Questionnaire (RPQ), Migraine Disability Assessment Test (MIDAS), Headache Impact Test (HIT-6), Beck Depression (BDI-II) and Anxiety Inventories, Post-Traumatic Stress Disorder Checklist—Civilian (PCL-5), and Pittsburgh Sleep Quality Index (PSQI).

Cognitive functioning was assessed across the domains of executive functioning, processing speed, attention, working memory, and episodic memory using CANTAB (www.cantab.com). Cognitive results for our cohort were compared to 35 healthy age-, sex-, and education-matched controls.

Blood testing assessed kidney and liver functions, fasting glucose and insulin levels, lipid panel, complete blood count, thyroid stimulating hormone, and C-reactive protein.

MRI was conducted using a 3-T GE MR750 MRI scanner and included T1, T2, diffusion-weighted, and dynamic contrast-enhanced imaging (DCE-MRI).

For Diffusion MRI analysis, we used Mrtrix3 software 12 and Fixel-Based Analysis. A total of 66 scans were analyzed: 18 from exposed subjects, 8 from non-exposed subjects within our study, and 40 from healthy age- and sex-matched controls.

Voxel-based blood-brain barrier (BBB) imaging analysis was performed as reported. A total of 37 scans were analyzed: 17 from exposed subjects, 11 from non-exposed subjects within our cohort including 6 that were analyzed both pre- and post-exposure, and 9 additional healthy age- and sex-matched controls.

Resting state MEG data were collected using an Elekta Neuromag whole head 306-channel MEG system. The inventors used fast Fourier transform (FFT) analysis for studying alterations in brain activity. Periods of paroxysmal slow wave events (PSWEs) were defined as time periods of brain activity in which the median power frequency was less than 6 Hz for more than 5 seconds. A total of 84 data sets were analyzed: 21 from exposed subjects, 12 from non-exposed subjects within our cohort, and 51 from non-exposed individuals outside our cohort, including 7 healthy non-exposed individuals recorded within the same MEG and additional 44 from healthy age- and sex-matched controls obtained from the CamCAN repository (www.mrc-cbu.cam.ac.uk/datasets/camcan). Seven subjects were analyzed both pre- and post-exposure.

### Need-Based Assessments

Individuals who reported symptoms suggesting brain injury underwent further neurological, visual, and audiovestibular assessments.

The neurological assessment included a clinical examination, Sport Concussion Assessment Tool (SCAT5), and the King-Devick Test.

The visual assessment included evaluation for afferent and efferent visual system defects and a detailed assessment of ocular alignment in all positions of gaze as well as horizontal saccadic velocities and fixation stability.

The audiovestibular assessment included pure tone testing, otoacoustic emissions and immittance testing, tympanometry and acoustic reflex threshold testing, auditory evoked potential testing, a Video Head Impulse Test (vHIT), Videonystamography (VNG), a caloric test, and cervical and ocular vestibular myogenic potential (CVEMP, OVEMP) testing.

### Statistical Analysis

Statistical data analysis was performed using MATLAB and R. Because some individuals were analyzed both before and after exposure while others were analyzed exclusively as exposed or exclusively as non-exposed, partial paired data analyses were used. For binary dependent variables (i.e., symptoms, questionnaires, toxicology), the partially overlapping samples z-test for proportions was performed. For parametric dependent variables (i.e., serum concentrations), the partially overlapping samples t-test was used. For non-parametric dependent variables, the rank-based two-sample test for paired data with missing values was performed. For fiber tractography, statistical analysis was conducted using Connectivity-based Fixel Enhancement (CFE), non-parametric permutation testing using 5000 permutations, and Family-Wise error correction at a P-value of 0.05. Age was included in the analysis as a nuisance covariate. For comparing damage levels between the exposed and non-exposed groups, a mask was generated for significant fixels, while extracted fiber density values were compared using the rank-based two-sample test for paired data with missing values. Statistical comparison of BBB leakage in subjects scanned prior to and shortly after exposure were performed using a paired two-tailed Wilcoxon signed rank test. P-values below 0.05 were considered significant.

### Example 1

### Slow Network Activity in AD is Composed of Distinct Paroxysmal Slow Waves Events

To explore changes in the function of brain networks, the inventors analyzed EEG recordings from patients attending the Cognitive Neurology Clinic at Rabin Medical Center, Beilinson Hospital, Petach Tikva, Israel and diagnosed with AD (N=16, mean age=72.11±9.67 years) or MCI (N=12, 73.37±4.76 years) compared with aged-matched controls (N=11, mean age=76.18, STD=10.61, see further clinical details in tables 1-3, hereinbelow). Spectral analyses revealed significantly lower relative power in alpha (8-12 Hz) among AD patients compared with MCI (p=0.003, Mann-Whitney, MW) or controls (p=0.006, MW). Consistent with previous reports AD patients showed significantly greater theta relative power (5-8 Hz) compared to age-matched controls (p=0.0147, MW, FIGS. 1A-1B). By studying the temporal characteristics of EEG slowing, the inventors found that it was composed of transient events, in which the network switched from apparently normal activity to brief periods of low frequency activity, which was termed “paroxysmal slow wave events” (PSWE, FIG. 1C). To further characterize PSWEs, the inventors calculated the median power frequency (MPF) in 2 sec-long signal epochs with 1 sec overlap and repeated the detection of PSWEs in a variety of upper-bounds (2-9 Hz with 1 Hz interval between iterations). For each iteration, the inventors used a blind gaussian mixture model (GMM) algorithm to cluster PSWE frequency (per minute) into high- and low-occurrence groups (HPSWE and LPSWE, respectively). ROC (receiver operating characteristic) analysis revealed that MPF of 6 Hz is the frequency that best separates AD patients and age-matched controls. Thus, an “event” was classified as a PSWE if the MPF is lower than 6 Hz for 5 consecutive seconds or more (FIG. 1D). The occurrence per minute of PSWEs was significantly higher in AD compared with aged-matched controls (P=0.001, MW) and with MCI patients (p=0.0015, MW). Nine of the 16 AD patients were classified into the HPSWE group, while only 1 of the 12 MCI patients and 1 of the 11 controls were classified to the HPSWE group. Thus, the prevalence of AD in the HPSWE group (90%) was significantly higher compared to their prevalence in LPSWE groups (41%, p=0.0159, Chi square, FIG. 1E). Additionally, mini-mental state examination (MMSE) scores among the HPSWE group was significantly lower compared to the LPSWE group (p=0.0034, MW, FIG. 1F). Moreover, MMSE score was significantly and inversely correlated with PSWE occurrence per minute (p=0.006, Pearson's correlation, FIG. 1G), suggesting that PSWEs reflect abnormal brain function, associated with cognitive impairment. Further characterizing the PSWEs, the inventors found that within the HPSWE group they were longer in duration compared to those in the LPSWE group (p<0.0001, MW, FIG. 1H). Heat scalp mapping showed that PSWEs were recorded in most scalp electrodes (FIG. 1I).

TABLE 1
Patients presented in FIG. 1.

| Character- | | | | | | |
|---|---|---|---|---|---|---|
| istics | Controls | MCI (n = 12) | | AD (n = 16) | | P value |
| Age (years) | 76.73 ± 10.06 | 73.71 ± 4.658 | | 72.11 ± 9.675 | | 0.54 |
| Female | 8 (72) | 7 | (58) | 6 | (37.5) | 0.18 |
| sex (%) | | | | | | |
| MMSE | | 28 | (27-29) | 20 | (14-26) | <0.0001 |

Age (mean and SD) was compared by Kruskal-Wallis test. Sex was compared by Chai square. MMSE (median and interquartile range) was compared by Mann Whitney.

TABLE 2
Patients with EEG recordings presented in FIG. 2.

| Characteristics | Controls (n = 9) | epilepsy | P value |
|---|---|---|---|
| Age (years) | 40 ± 14.09 | 32.41 ± 14.73 | 0.18 |
| Female sex (%) | 7 (78%) | 8 (47%) | 0.13 |

Age (mean and SD) was compared by Mann-Whitney test. Sex was compared by Chai square.

TABLE 3
Patients with DCE-MRI scan in FIG. 2.

| Characteristics | Controls (n = 60) | epilepsy | P value |
|---|---|---|---|
| Age (years) | 28.52 ± 4.02 | 28.33 ± 13.85 | 0.44 |
| Female sex (%) | 20 (33.3%) | 6 (50%) | 0.27 |

Age (mean and SD) was compared by Mann-Whitney test.

### Example 2

### PSWEs in Patients with Epilepsy

Comorbidity of AD and epilepsy is attracting increased attention due to the promise of anti- epileptic therapy for some AD patients. Since PSWEs are paroxysmal and transient changes in network activity, similar to the characteristics of epileptic seizures, the inventors next analyzed EEG from patients with epilepsy (N=17, mean age=32.41 years, STD=14.73) in comparison with age-matched controls (N=9, mean age=40.67 years, STD=14.09) and found PSWEs more frequent in epilepsy patients (p=0.0052, FIG. 2A). When grouped to high (>mean controls+2 standard deviations, HPSWE) and low (LPSWE) PSWE groups, the inventors found that epilepsy patients comprised 90% of the HPSWE group (9/10) and 47% (8/17) in the LPSWE group (p=0.0257, Chi square, FIG. 2B). To challenge the hypothesis that PSWEs are associated with BBBD regardless of other age-related effects that can be found in the AD patients the inventors implemented DCE-MRI to test BBBD in epilepsy patients. In 12 patients with epilepsy (mean age=28.33, STD=13.86) and 60 age-matched controls (age=28.52 years, STD=4.027, Table 3) BBBD was significantly greater among epilepsy patients compared with controls (p=0.0056, MW, FIG. 2C). When extent of BBBD (percent brain voxels with pathological permeability) was grouped into low (LBBBD) and high (HBBBD) groups (below and above mean plus 2 STD of control values) the inventors found that the prevalence of epilepsy patients among the HBBBD group was significantly greater compared with the LBBBD group (50%, (4/8) vs. 12.5% (8/64), respectively, p=0.0073, Chi square, FIG. 2D). In a subset of epilepsy patients (N=10) both DCE-MRI and EEG were conducted. The percentage of brain volume with BBBD was calculated separately for 8 cortical lobes (Left and right frontal, temporal, parietal, and occipital) and correlated with PSWEs occurrence per minute as detected in electrodes corresponding to the same lobe. In 8 of 10 patients positive spearman's r values were found (FIGS. 6E-6G).

### Example 3

### PSWEs and BBBD Characterize Aged Mice

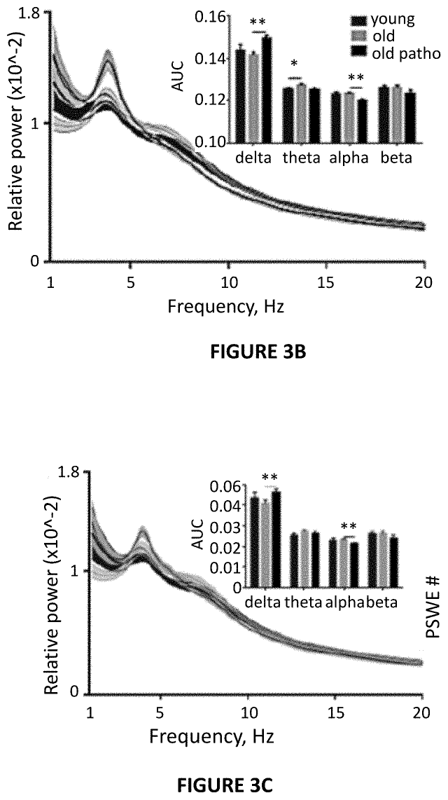

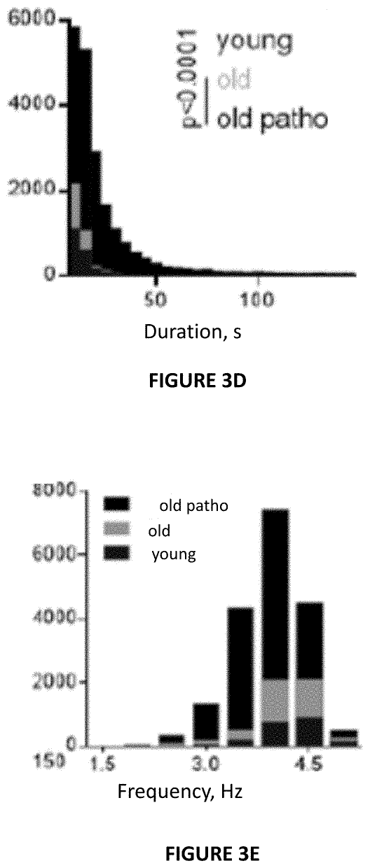

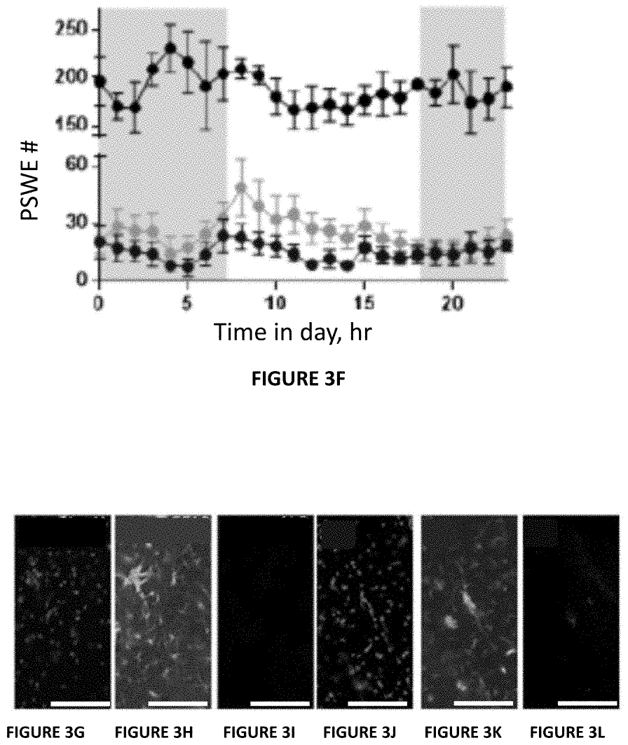

The inventors next compared brain activity recorded from aged mice (N=12, 18-22 months old), a natural model of age-related cognitive decline27, to that of young animals (n=5, 12 weeks of age). Similar to the findings in humans, the inventors detected PSWEs, and found that frequency threshold of 5 Hz and minimal duration of 10 s yield optimal separation of old animals into 2 groups by GMM [old LPSWE vs. old HPSWE, FIG. 3A]. Spectral analysis showed significantly greater delta (p=0.0022, MW) and lower alpha relative power (p=0.0022, MW) in recordings from old HPSWE compared with old LPSWE. Old LPSWE animals had significantly greater theta relative power compared with young (p=0.04, MW, FIG. 3B). In line with the human findings in AD patients, the inventors found that PSWEs in the old HPSWE group were significantly longer compared with PSWEs among young (p<0.0001, MW) or old LPSWE groups (p<0.0001, MW). Median frequency of PSWEs was significantly lower among old HPSWE group compared with old LPSWE or young groups (p<0.0001, MW, FIG. 3E). As slow activity is known to occur during deep sleep, the inventors examined PSWEs occurrence throughout a 24 h cycle. The number of PSWE among the old HPSWE group was significantly higher regardless the time of the day (p<0.0001 for each hour, multiple t tests, FIG. 3F). Analysis of locomotor activity confirmed that aged animals were moving for more than 75% of the time during PSWEs, suggesting these are not sleep-related episodes (FIG. 6). Finally, to test if the old animals show BBBD and glial activation, the inventors performed immunohistochemistry (IHC) stains for NeuN, GFAP and albumin for brains of young (N=3, 3 months of age) and old mice (N=4, 21 months of age). Indeed, GFAP and albumin staining was found greater among old mice compared with young (FIGS. 3G-3L).

### Example 4

### PSWE and BBBD in a Status Epilepticus Model of Epilepsy

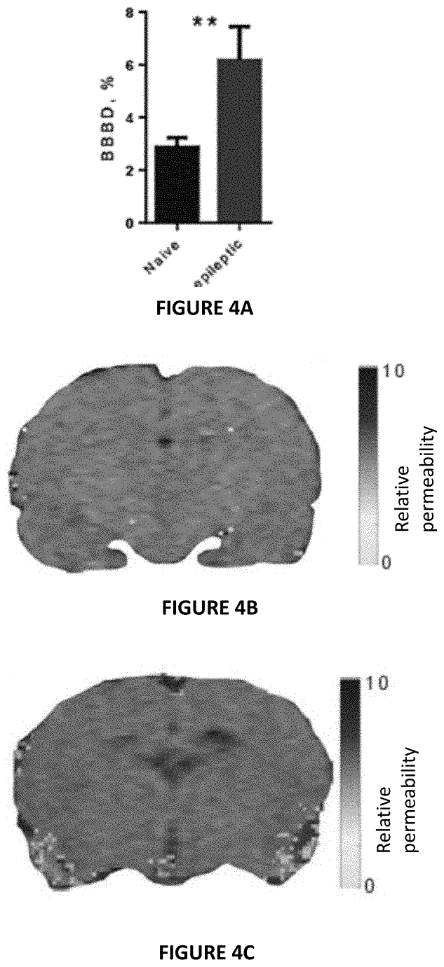

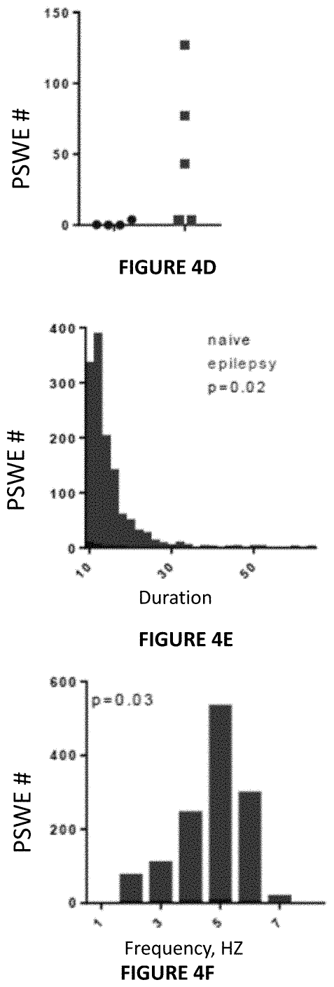

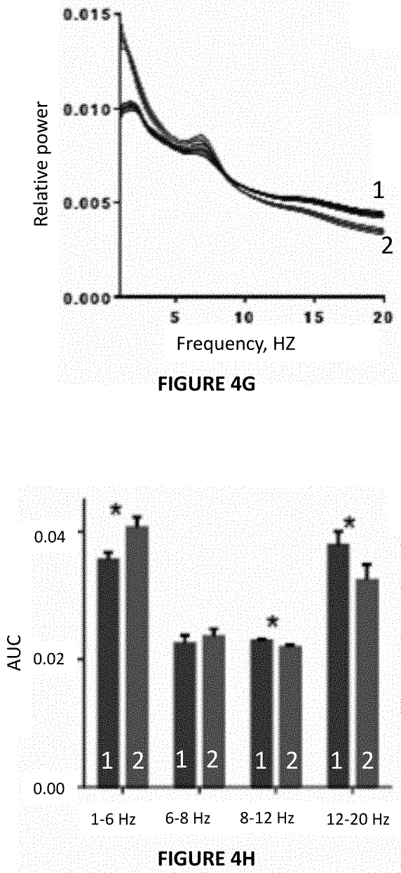

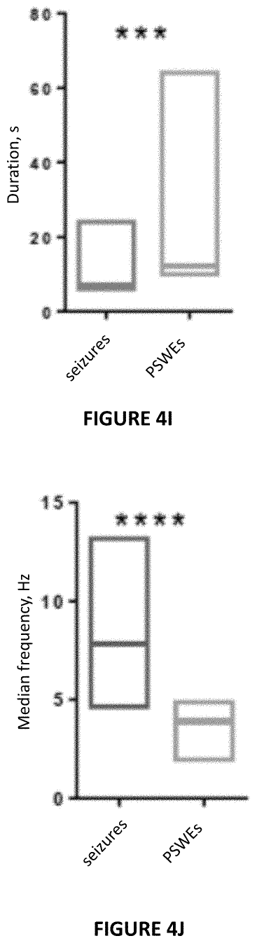

Consistent with the inventors observations in patients with epilepsy, direct recordings from epidural electrodes implanted in young rats (3 months of age) with SE-induced epilepsy showed higher occurrence of PSWEs (p=0.055, MW), with a longer duration (p<0.02, MW) and lower median frequency (p<0.03, MW) in epileptic compared with naïve animals (FIGS. 4D-4F). ECoG spectral analysis revealed relative power greater in delta (1-5 Hz, p=0.0357, MW) and lower alpha (8-12 Hz, p=0.0357, MW) and beta (12-20 Hz, p=0.0357, MW) among epileptic rats compared with naïve (FIGS. 4G-4H). Finally, the inventors compared frequency and duration of PSWEs with that of identified convulsive epileptic seizures (detected automatically). Spontaneous seizures (N=18, Median=7 s, min=6 s, max=24 s) were significantly shorter (p=0.0007, MW) than PSWEs (N=4885, median=12 s, min=10 s, max=64 s, FIG. 4I), and with a higher median frequency (p<0.0001, MW, spontaneous seizures: median=7.813, min=4.639, max=13.18 Hz, PSWE: median=3.418, min=1.953, max=4.883 Hz, FIG. 4J) compared with PSWEs. Animals with epilepsy also demonstrated persistent cortical BBBD, as confirmed using contrast-enhanced MRI (FIGS. 4A-4C), further supporting the association between BBBD and PSWEs.

### Example 5

### Brain Exposure to Serum Albumin Induces PSWE Network Activity

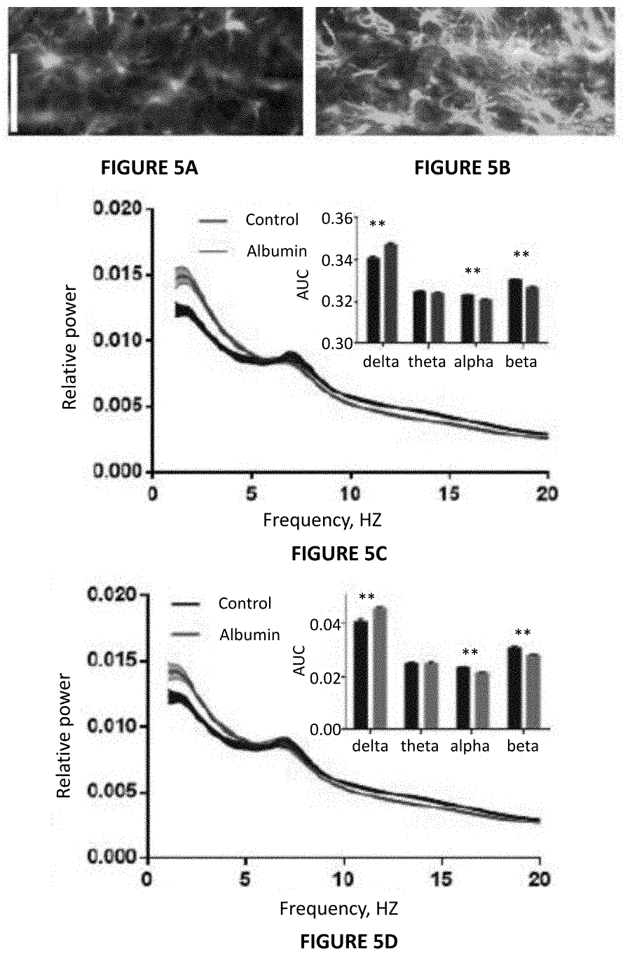

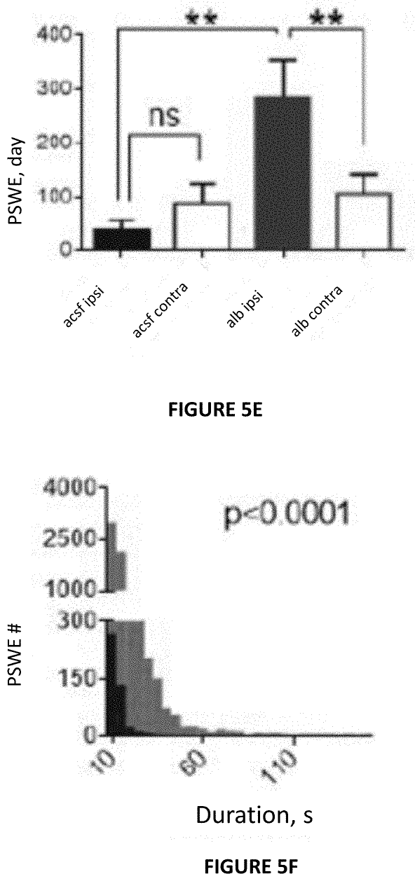

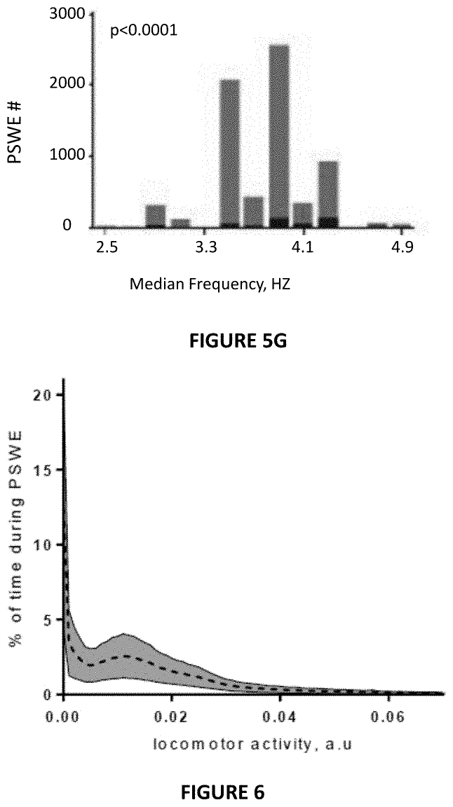

Finally, as BBBD has been suggested to have a role in the pathogenesis of both AD and epilepsy, the inventors tested the causal role of BBBD and specifically, the serum protein albumin in network dysfunction. The inventors used osmotic pumps in rats to inject albumin into the right lateral ventricle (ICV) for 28 days and performed IHC to confirm the accumulation of albumin in brain astrocytes (FIGS. 5A-5B), similar to that observed in old mice (FIGS. 3G-3L). The inventors next recorded ECoG from young rats (10-12 weeks of age) exposed to ICV albumin (N=8), compared to animals exposed to ACSF (controls, N=5). Four weeks after onset of perfusion (but not at one week), ECOG analysis detected significantly higher occurrence per minute of PSWEs in the albumin- injected hemisphere compared with the non-injected hemisphere (p=0.0078, MW) or ACSF-injected in controls (p=0.0070, MW, FIG. 5E). The albumin-treated group also showed significantly greater relative power in the delta (1-5 Hz) band compared with ACSF-injected animals (p=0.0016, MW), and significantly lower relative power in alpha (8-12 Hz, p=0.0016, MW) and beta (12-20 Hz, MW, p=0.0031) bands (FIG. 5C). Consistently with the findings in patients and animal models, PSWEs in the albumin treated group were significantly longer (p<0.0001, MW, FIG. 5F) and slower (p<0.0001, MW, FIG. 5G) compared with the ACSF treated group.

### Example 6

### Bipolar Patients

A cohort of 36 bipolar patients was recruited for the study (23 bipolar type I and 13 bipolar type II). The average duration of illness among the patients was 28±13 years, with an average onset at 22±10 years of age. Bipolar disorder started with a depressive episode in 76% of patients, and about half (55%) have progressed to a chronic course of illness. The average age of the group was 49.1±11.3 years and 70.6% were females. Control subjects were matched for sex, age, and metabolic syndrome (Table 4). Compared to controls, bipolar patients scored significantly worse on scales of depression (Montgomery-Á̊sberg depression rating scale), anxiety (Hamilton anxiety rating scale), and capacity of carrying out day-to-day functions (Global Assessment of Functioning, Table 4). No differences in anthropometric or metabolic measures were identified between the groups (Table 4).

TABLE 4
Participant characteristics

| | Bipolar Patients | | Controls | | P value |
|---|---|---|---|---|---|
| Demographics | | | | | |
| Age | 49.1 | (1.9) | 47.6 | (2.9) | 0.666 |
| Sex (% female) | 70.6 | | 71.4 | | 1.000 |
| Anthropometric and metabolic measures | | | | | |
| Body mass index (BMI) | 30.1 | (1.1) | 28.2 | (1.5) | 0.358 |
| Waist-to-hip ratio | 0.9 | (0.02) | 0.9 | (0.03) | 0.230 |
| Risk of cardiovascular disease | 8.6 | (1.5) | 4.9 | (0.8) | 0.469 |
| (Framingham risk score) | | | | | |
| Framingham heart age | 53.0 | (5.3) | 47.7 | (2) | 0.602 |
| Metabolic syndrome (% subjects) | 27.8 | | 15.4 | | 0.474 |
| Insulin resistance (HOMA-IR score) | 2.7 | (0.3) | 1.7 | (0.2) | 0.056 |
| Psychiatric characteristics | | | | | |
| Depression severity (MADRS score) | 18.1 | (2.4) | 1.9 | (0.4) | <0.001 |
| Anxiety severity (HAM-A score) | 11.8 | (1.5) | 2.0 | (0.4) | <0.001 |
| Global Assessment of Functioning | 66.8 | (0.5) | 92.1 | (0.7) | <0.001 |
| (GAF score) | | | | | |
| Medication use (% patients) | | | | | |

| | | | |
|---|---|---|---|
| Lithium | 72 | — | — |
| Antiepileptics | 67 | — | — |
| Atypical antipsychotics | 56 | — | — |
| Antidepressants | 44 | — | — |
| Benzodiazepines | 56 | — | — |
| Melatonin | 19 | 0 | 0.169 |
| Blood pressure medication | 14 | 14 | 1.000 |
| Cholesterol medication | 14 | 0 | 0.304 |

Mean (standard error), unless otherwise indicated. Continuous variables were compared using the Wilcoxon rank sum test, and categorical variables were compared using Fisher's Exact Test. MADRS, Montgomery-Á̊sberg depression rating scale; HAM-A, Hamilton anxiety rating scale; HOMA-IR, homeostatic model assessment of insulin resistance.

### Example 7

### Extensive BBB Leakage is Associated with Greater Psychiatric Morbidity in Bipolar Patients

All participants underwent dynamic contrast enhanced MR imaging, and quantitative maps of BBB permeability were calculated based on contrast accumulation-rates in each brain voxel (FIG. 7A). The percent of brain tissue affected by BBB leakage was used as a global measure of BBB dysfunction, revealing a high variability of values among the bipolar cohort (FIG. 7B). Blinded K-means cluster analysis of all 50 participants identified a sub-group of ten subjects with significantly higher levels of BBB dysfunction (p<0.0001, FIG. 7C). These were identified as subjects with over 12.75% of the brain affected by leakage and were labelled the “extensive BBB leakage” group. Notably, this group consisted exclusively of bipolar patients. The group with the lower levels of BBB dysfunction included the entire control cohort as well as the remaining 26 bipolar patients. Since there were no differences between the controls and patients within this group, it was collectively referred to as the “normal BBB leakage” group. To examine whether the differences between bipolar patients with extensive vs. normal leakage were widespread (diffuse) or restricted to specific brain regions (focal), the extent of BBB leakage was quantified in 126 anatomically/functionally significant brain regions and compared between the two groups. The comparison revealed a diffuse rather than focal difference, with 112 of the 126 regions showing significantly higher leakage in the “extensive BBB leakage” group (FIG. 7D, p<0.05, corrected for multiple comparisons). The length and severity of manic/depressive episodes was also found to be different between the two groups, with higher rates of chronic versus episodic course of bipolar illness among patients with extensive BBB leakage (FIGS. 7E-7F). Moreover, extensive BBB leakage was found to be associated with increased depression, more severe anxiety, and reduced social and occupational functionality (FIG. 7F). No associations between BBB pathology and age, disease duration or cognitive dysfunction were found in our cohort.

### Example 8

### Extensive BBB Leakage is Associated with Metabolic Dysregulation, Yet not with Class of Mood-Stabilizing Drugs

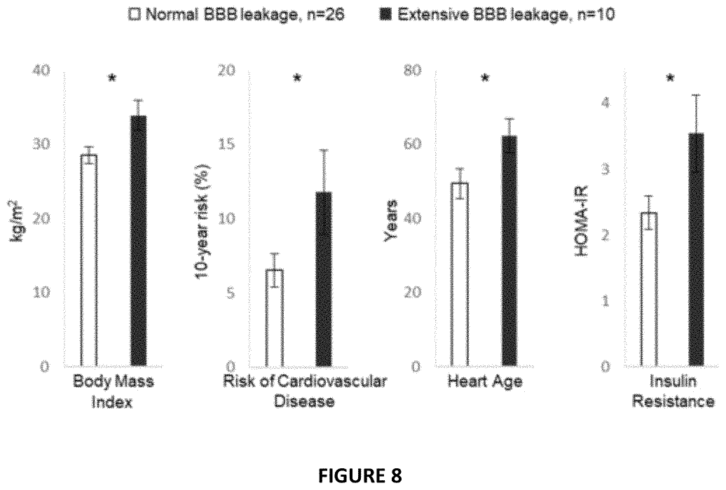

Bipolar patients with extensive BBB leakage were found to have higher body-mass indices, elevated risk of cardiovascular disease, and advanced heart age (FIG. 8), Furthermore, all patients within the “extensive BBB leakage” group were also found to have comorbid insulin resistance (homeostatic model assessment of insulin resistance >1.8). Notably, while all subjects with extensive BBB leakage had insulin resistance, not all subjects with insulin resistance had extensive BBB leakage (with four insulin resistant controls and 12 insulin resistant bipolar patients having normal levels of BBB leakage). No patients were receiving anti-diabetic or insulin sensitizing drugs. No differences in the class of mood stabilizing treatments were found between the normal and extensive BBB leakage groups.

### Example 9

### Aberrant Paroxysmal Slow Wave Events in Aged Mice

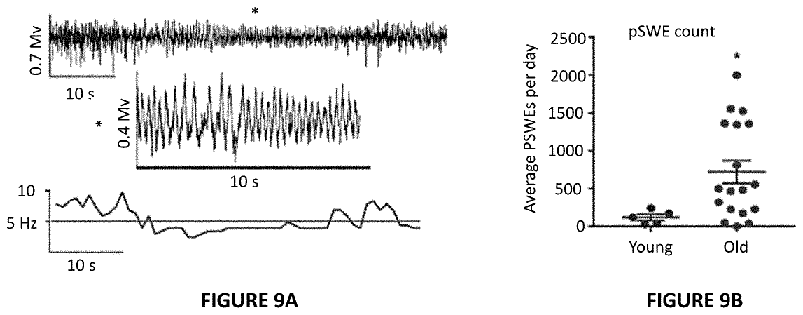

Next, the inventors sought to measure and characterize hyperexcitability recording telemetric electrocorticography (ECOG) using epidural electrodes implanted in young (3 months old) and old (18 to 24 months old) mice over a period of 5 days in the home cage. The inventors found that aged mice showed an increase in the relative power of slow wave activity (<5 Hz) (data not shown), which is thought to reflect dysfunctional neural networks. Detailed analysis of this aberrant ECOG signal revealed that the slow wave activity was not continuous, but rather manifested in discrete, transient paroxysmal slow wave events (PSWEs) (median frequency <5 Hz; FIG. 9A), which were elevated in aged mice relative to young mice (FIG. 9B).

### Example 10

### Havana Syndrome Study—Environmental Exposure to Neurotoxins Affect the Brain'S Cholinergic System

Symptoms among the exposed included cognitive impairment (impaired concentration and memory), visual impairment (blurred vision and sensitivity to light), audiovestibular impairment (tinnitus, sensitivity to sound, feeling off balance), and generally reduced well-being (sleep disturbances, fatigue, headaches, irritability). In case of anthropometric measures and blood tests, no significant differences were found between exposed and non-exposed groups. In self-reported symptom questionnaires, of 8 questionnaires, 3 were scored positively by the majority of exposed individuals: RPQ (for post-concussive syndrome), HIT-6 (for headache severity), and MIDAS (for migraine). In cognitive assessments: the inventors recorded lower performance in spatial working memory among exposed subjects compared to both non-exposed subjects and age-, sex-, and education-matched controls (P=0.017). A milder reduction in performance was also found in decision-making quality (P=0.057). See tables 5-8 herein below.

TABLE 5
clinical and research measures used
Initial Screening

| | |
|---|---|
| Anthropometric | Height, weight, blood pressure, heart rate |
| measures | |
| Self-rated | Rivermead Post-Concussion Symptoms Questionnaire |
| symptom | Migraine Disability Assessment (MIDAS) |
| questionnaires | Headache Impact Test (HIT-6) |
| | Pittsburgh Sleep Quality Index (PSQI) |
| | Beck Depression (BDI-II) |
| | Beck Anxiety Inventory (BAI) |
| | Post-Traumatic Stress Disorder Checklist - Civilian (PCL-5) |
| Cognitive tests | Executive functions; processing speed; attention; working |
| | memory and episodic memory using CANTAB |
| | (www.cantab.com). |
| Laboratory tests | Kidney and liver functions, fasting glucose and insulin levels, |
| | lipid panel, complete blood count, thyroid stimulating hormone, |
| | C-reactive protein. |
| | To investigate cholinesterase-inhibition hypothesis: AChE, |
| | BChE, toxicology |
| MRI | T1, T2, DWI, resting fMRI, DCE-MRI |
| MEG | Resting. Eyes closed and open. |
| Need-based | |
| Assessments | |
| Neurological | Clinical examination, Sport Concussion Assessment Tool, 5th |
| | Edition (SCAT5), and the King-Devick Test, a clinical test of eye |
| | movements used to screen for concussions |
| Vision | General afferent visual function testing, orthoptic evaluation, eye |
| | tracking (fixation stability and saccades), examination of fundus |
| | (photographs) |
| Audiovestibular | Audio: pure tone testing, high-frequency (HF) testing, |
| | otoacoustic emissions test, tympanometry and acoustic reflex |
| | thresholds, auditory evoked potentials |
| | Vestibular: Videonystagmography (Caloric, Oculomotor, |
| | Positional, Positioning), Cervical and Ocular Vestibular Evoked |
| | Myogenic Potentials, Video Head Impulse Test, Dizziness |
| | Handicap Inventory (DHI), Activities Specific Balance |
| | Confidence Scale (ABC) |

TABLE 6
prevalence of persistent symptoms

| | | | | | | Estimated | |
|---|---|---|---|---|---|---|---|
| | | Non- | | Exposed | | Difference in | P-Value |
| | | Exposed | | N = 23 | | Proportions | (z- |
| Domain | Symptom | N = 12 (%) | | (%) | | [95% CI] | statistic) |
| Cognitive | Concentration | 0 | (0) | 10 | (43.5) | 0.435 | 0.007 |
| | | | | | | [0.119, 0.750] | (2.70) |
| | Memory | 0 | (0) | 9 | (39.1) | 0.391 | 0.012 |
| | | | | | | [0.086, 0.696] | (2.51) |
| Dizziness | Balance | 0 | (0) | 13 | (56.5) | 0.565 | 0.001 |
| | | | | | | [0.228, 0.902] | (3.28) |
| | Vertigo | 1 | (8.3) | 7 | (30.4) | 0.221 | 0.159 |
| | | | | | | [−0.086, 0.528] | (1.41) |
| | Light | 0 | (0) | 6 | (26.1) | 0.261 | 0.052 |
| | Headedness | | | | | [−0.002, 0.524] | (1.94) |
| Visual | Blurred | 1 | (8.3) | 10 | (43.5) | 0.351 | 0.019 |
| | Vision | | | | | [0.057, 0.646] | (2.34) |
| | Light | 0 | (0) | 8 | (34.8) | 0.348 | 0.020 |
| | Sensitivity | | | | | [0.055, 0.641] | (2.33) |
| Audiovestibular | Tinnitus | 0 | (0) | 9 | (39.1) | 0.391 | 0.012 |
| | | | | | | [0.086, 0.696] | (2.51) |
| | Sound | 0 | (0) | 10 | (43.5) | 0.435 | 0.007 |
| | Sensitivity | | | | | [0.119, 0.750] | (2.70) |
| | Vestibular | 0 | (0) | 7 | (30.4) | 0.304 | 0.033 |
| | | | | | | [0.025, 0.584] | (2.14) |
| General Well- | Sleep | 1 | (8.3) | 14 | (60.9) | 0.525 | 0.003 |
| Being | | | | | | [0.180, 0.871] | (2.98) |
| | Fatigue | 0 | (0) | 13 | (56.5) | 0.565 | 0.001 |
| | | | | | | [0.228, 0.902] | (3.28) |
| | Headaches | 0 | (0) | 17 | (73.9) | 0.739 | <0.001 |
| | | | | | | [0.390, 1.09] | (4.15) |
| | Irritability | 0 | (0) | 6 | (26.1) | 0.261 | 0.052 |
| | | | | | | [−0.002, 0.524] | (1.94) |
| | Nausea | 3 | (25) | 9 | (39.1) | 0.141 | 0.303 |
| | | | | | | [−0.128, 0.410] | (1.03) |

TABLE 7
self-reported symptom questionnaires

| | | | | | | Estimated | |
|---|---|---|---|---|---|---|---|
| | | | | | | Difference | |
| | | Non- | | | | in | P-Value |
| | | Exposed | | Exposed | | Proportions | (z- |
| Domain | Questionnaire | N = 12 (%) | | N = 23 (%) | | [95% CI] | statistic) |
| Concussion | RPQ | 2 | (16.7) | 13 | (56.5) | 0.399 | 0.011 |
| | | | | | | [0.090, 0.707] | (2.54) |
| Headaches | HIT-6 | 0 | (0) | 13 | (56.5) | 0.565 | 0.001 |
| | | | | | | [0.228, 0.902] | (3.28) |
| Sleep | PSQI | 7 | (58.3) | 14 | (60.9) | 0.053 | 0.715 |
| | | | | | | [−0.231, 0.337] | (0.366) |
| Migraine | MIDAS | 0 | (0) | 7 | (30.4) | 0.304 | 0.033 |
| | | | | | | [0.025, 0.584] | (2.14) |
| Mental State | MMS | 0 | (0) | 3 | (13.0) | 0.130 | 0.191 |
| | | | | | | [−0.065, 0.326] | (1.31) |
| Depression | BDI-II | 0 | (0) | 4 | (17.4) | 0.174 | 0.125 |
| | | | | | | [−0.048, 0.396] | (1.54) |
| Anxiety | BAI | 0 | (0) | 2 | (8.7) | 0.087 | 0.293 |
| | | | | | | [−0.075, 0.249] | (1.05) |
| PTSD | PCL-5 | 0 | (0) | 2 | (8.7) | 0.087 | 0.293 |
| | | | | | | [−0.075, 0.249] | (1.05) |

TABLE 8
Cognitive Assessment Results

| | | Paired | Spatial | | Stockings | Cambridge |
|---|---|---|---|---|---|---|
| | Reaction | Associates | Working | Multitesting | of | Gambling |
| Test | Time¹ | Learning² | Memory³ | Task⁴ | Cambridge ⁵ | Task⁶ |
| Non- | 370.8 ± 34.3, 47 | 9.19 ± 8.40, 47 | 2.57 ± 4.64, 35 | 3.00 ± 2.67, 46 | 6.14 ± 1.23, 41 | 0.985 ± 0.019, 35 |
| Exposed | | | | | | |
| (M ± SDV, N) | | | | | | |
| Exposed | 371.6 ± 25.3, 20 | 8.55 ± 9.30, 20 | 8.11 ± 7.33, 19 | 4.21 ± 4.14, 19 | 5.63 ± 0.71, 17 | 0.947 ± 0.064, 9 |
| (M ± SDV, N) | | | | | | |
| P (z) | 0.876 (0.153) | 0.650 (−0.455) | 0.017 (2.39) | 0.267 (1.11) | 0.182⁷ | 0.057 (−1.90) |

¹Median five choice reaction time;
²Total Errors (adjusted);
³Number of errors (across all);
⁴Number of incorrect responses (total);
⁵ Mean number of moves;
⁶Decision-making quality (total score);
⁷Wilcoxon test

Need-Based Assessments—Neurological Assessment: No neurological deficits were found on clinical examination; Visual Assessment: No consistent pathology among the exposed group, and no significant differences between exposed and non-exposed groups. Audiovestibular Assessment: Hearing loss was found in 3 of the 20 exposed subjects tested (15%) but was asymptomatic, and in 2 of the 3, could be attributed to a history of sound exposure prior to their stay in Havana. Auditory brain-stem evoked potentials, however, showed long latencies (both absolute and interpeak) in the majority of exposed individuals. Acoustic reflex was also found to be positive in 80% of exposed individuals. The most consistent finding in the vestibular assessment was the presence of low-threshold, high-amplitude cervical and/or ocular vestibular evoked myogenic potentials. This was found in up to 40% of exposed individuals. Both auditory and vestibular assessments were consistent with brain-stem dysfunction in the exposed group.

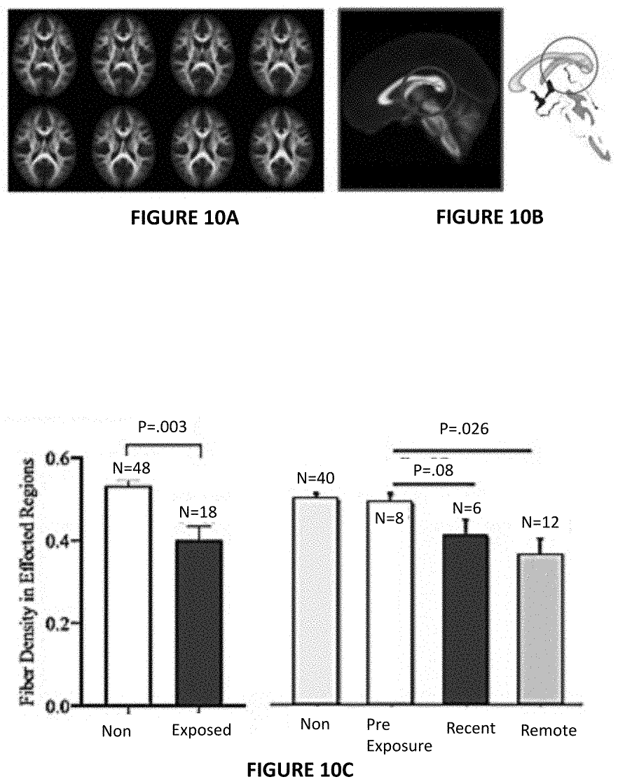

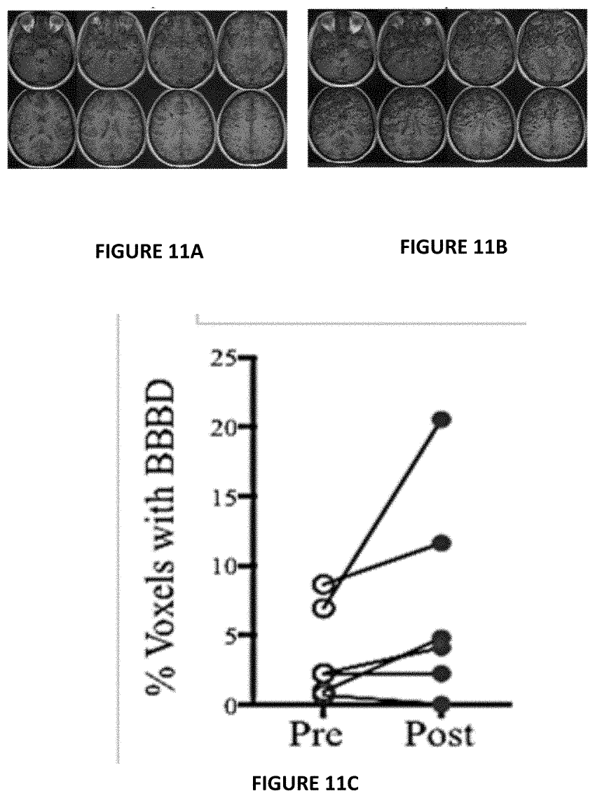

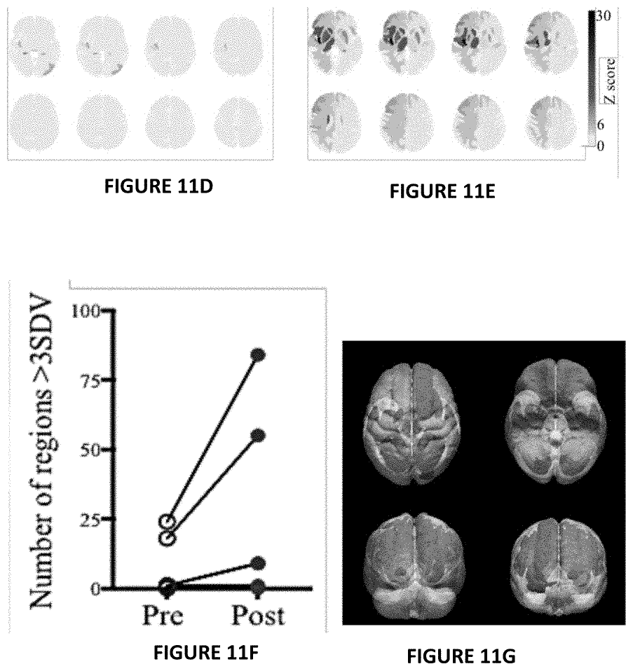

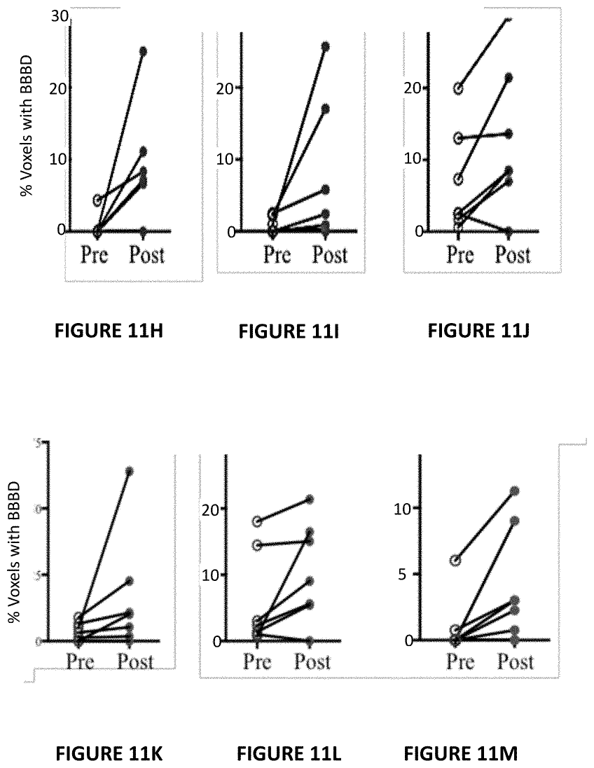

Brain Imaging (MRI & MEG)—Clinical MRI: In 4 of the 26 scanned subjects (all of whom were exposed), non-specific white matter hyperintensities in T2 and FLAIR sequences were observed; in another exposed subject, a small capillary telangiectasia in the pons was observed; in yet another exposed subject, Chiari I malformation was observed. Extracranial findings in 4 other (yet exposed) individuals included mucosal inflammatory changes of the paranasal sinuses. All such findings were considered incidental, without known clinical significance. Diffusion Tensor Imaging (MRI): A significant difference in white matter integrity between exposed and non-exposed groups was indicated. Decreased fiber density was observed in the exposed group along the right crus of the fornix, past the hippocampal commissure, and projecting into the hippocampus, as well as in the splenium of the corpus callosum (P=0.003). While there was a tendency for lower fiber density in the remotely exposed group, this was not found to be significant (FIG. 10). Dynamic Contrast-Enhanced MRI: Analysis of 6 subjects tested both before and after living in Havana revealed an increase in the volume and number of brain regions with a leaky blood-brain barrier (BBB). Statistical comparison of the extent of BBB dysfunction in 126 anatomical regions revealed 6 shared brain regions that were leakier after- compared to before-exposure scans (P<0.05). A leaky BBB was mainly found in the right hemisphere and included the right basal forebrain, anterior insula, posterior orbital, superior frontal, and superior occipital gyri (FIG. 11).

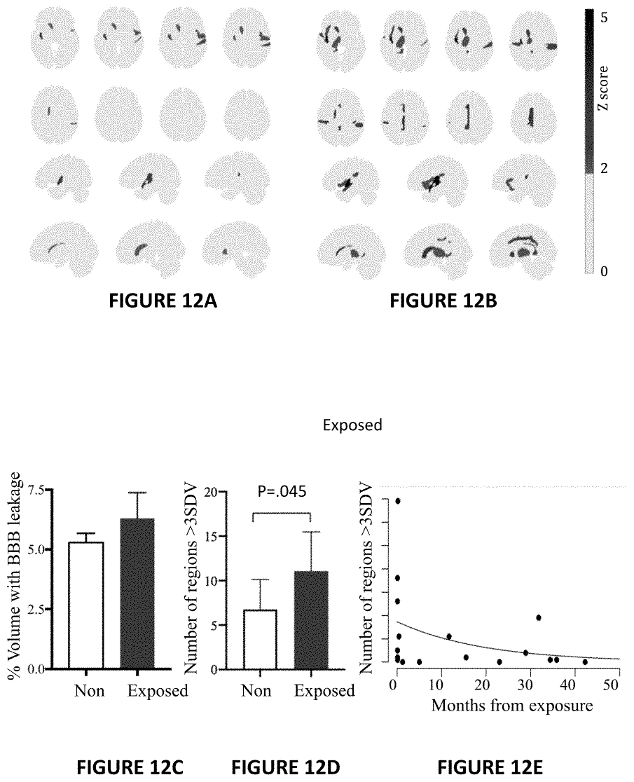

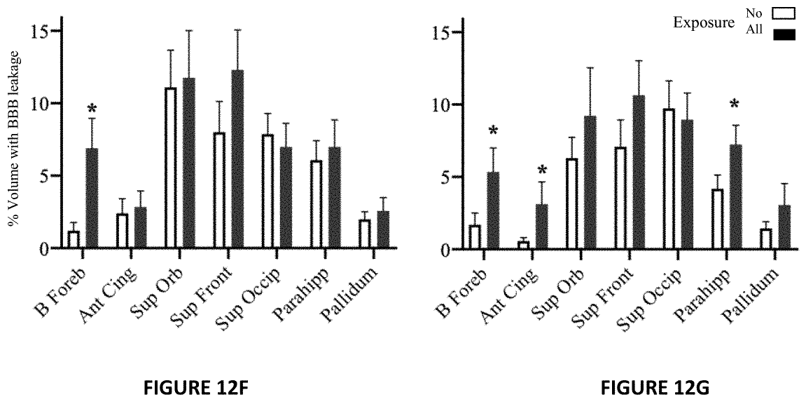

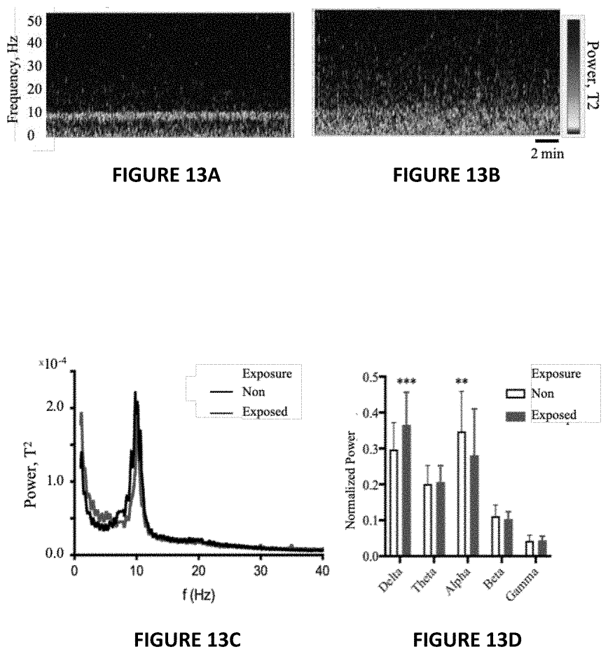

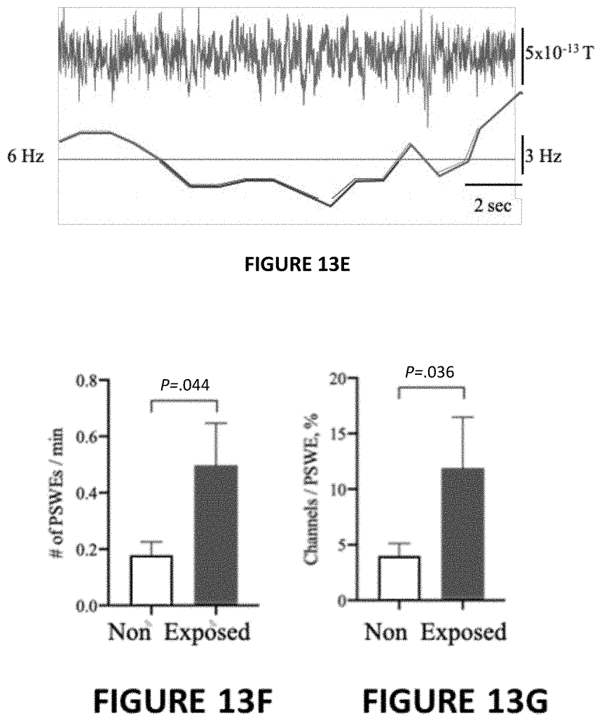

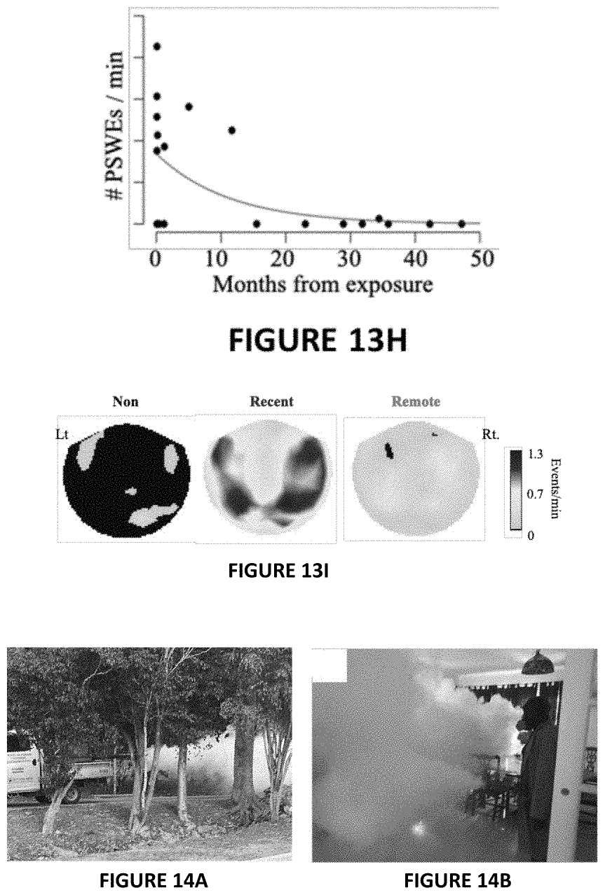

The inventors next compared regional BBB integrity between exposed and non-exposed groups (FIG. 12A-12B). While no differences were found in the total brain volume with a leaky BBB, the exposed group had more regions with a leaky BBB compared to non-exposed subjects and healthy controls (P=0.045, FIG. 12D). Furthermore, a zero-inflated Poisson regression showed a negative relationship between the number of regions with a leaky BBB and time away from Havana, although this was not significant (FIG. 12E). When the regions found to have changed in the post- compared to pre-exposure individuals were tested for the entire exposed group (FIGS. 11G-11M), the basal forebrain (bilaterally), right anterior insula, and parahippocampal gyrus were found to be significantly leakier in the exposed group compared to the non-exposed group and healthy controls (FIGS. 12F-12G). Magnetoencephalography (MEG): Exposed individuals showed a power increase in the delta frequency range as well as a decrease in the alpha frequency range (FIGS. 13A-13D). These changes were due to a transient, intermittent slowing of brain activity, termed “paroxysmal slow-wave events” (PSWEs, FIG. 4D). PSWEs were rarely detected among healthy controls, including those tested prior to exposure. By contrast, PSWEs were more common in both recently and remotely exposed groups (FIGS. 13F-13G). A Tweedie GLM log regression revealed a significant negative relationship between the number of PSWEs and time away from Havana (FIG. 13H). PSWEs were distributed in both hemispheres, although more prominently in the right hemisphere and in the recently exposed group, on (FIG. 13I).

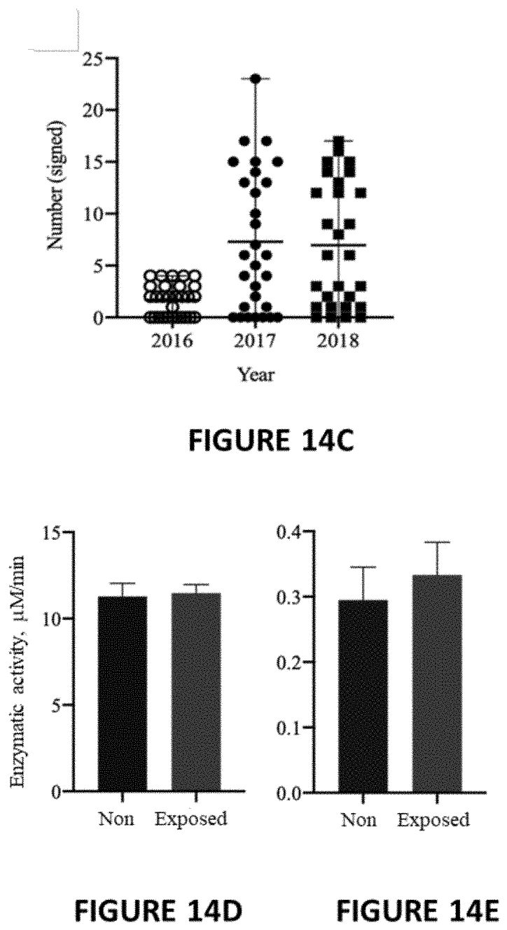

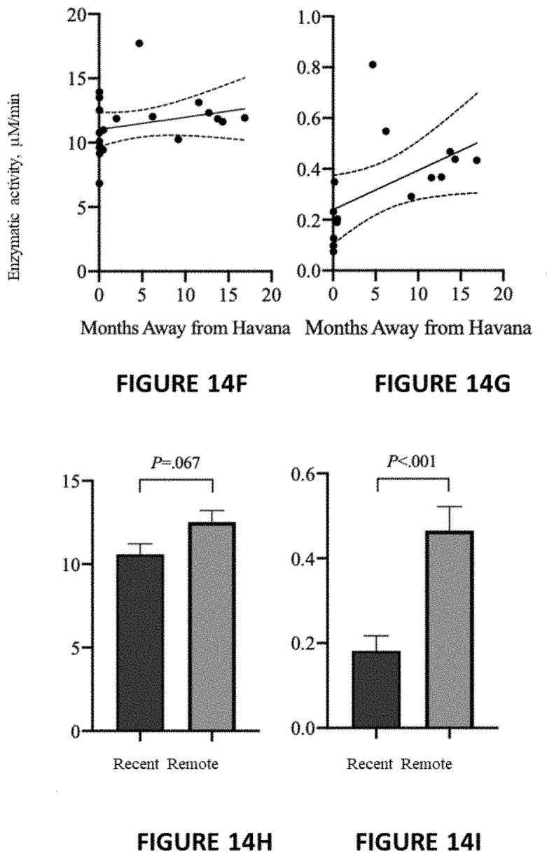

The herein disclosed findings—in particular (1) the involvement of cholinergic basal forebrain nuclei as found in BBB imaging, (2) reduced fiber density along the fornix, a fiber that includes cholinergic fibers leading from the basal forebrain nucleus to the hippocampus, (3) audiovestibular evidence suggestive of brain-stem dysfunction, and (4) diffuse cortical dysfunction as found on MEG recordings—indicated dysfunction in the brain's cholinergic system, and raised the hypothesis that one or more environmental neurotoxins targeting the cholinergic system may underlie the observed injury. The inventors had tested serum samples for acetyl- and butyryl-cholinesterase activity among the herein disclosed cohort. While there was no significant difference in activity between exposed and non-exposed groups overall (FIGS. 14D-14E), the inventors found that enzyme activity tended to increase over time since exposure among the exposed group (FIGS. 14F-14G). Among recently exposed individuals, furthermore, AChE and BChE activity was significantly reduced, further supporting enzymatic inhibition (FIGS. 14H-14I).

Mass-spectrometry further confirmed the presence of cholinesterase-inhibiting insecticides among exposed subjects, including Temephos, an organophosphorus insecticide used in Cuba against mosquito larvae, and 3-phenoxybenzoic acid (3-PBA), a common pyrethroid (insecticidal) metabolite. In particular, Temephos was detected in 9 (45%) of exposed individuals, compared to 2 (5.7%) of the controls (P<0.001). 3-PBA was found in the majority (65%) of exposed individuals, with no significant difference between the groups (data not shown).

Using a multimodal, quantitative, and control-tested approach, the herein disclosed results confirm brain injury, specify the regions involved, and suggest a likely etiology in the form of environmental exposure to neurotoxins affecting the brain's cholinergic system. Specifically, the inventors suggest that insecticides are likely to be a source. Though other sources of neurotoxins are possible, the herein disclosed insecticidal hypothesis gains contextual support given Cuba's well-documented efforts to aggressively mitigate the spread of the Zika virus by means of mass indoor and outdoor fumigations in 2016 and thereafter (FIGS. 14A-14B). Canadian Embassy records further confirmed a significant increase in the frequency of fumigations around and within staff houses, beginning January 2017, concurrent with reported symptoms (FIG. 14C).

Though most subjects in the herein disclosed study did not report an acute event of pressure or sound, in contrast to reports of American diplomats residing in Cuba, symptoms were similar to those reported, including headaches and difficulties with concentration, balance, vision, and sleep. The shared symptoms, location, period, and relative duration of time, all point to a shared etiology.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for determining blood-brain barrier dysfunction (BBBD) in a subject, comprising determining a paroxysmal slow waves event (PSWE) in said subject, wherein said PSWE has a median power frequency (MPF) of 3-10 Hz and is at least 5 seconds long, thereby determining BBBD in the subject.

2. The method of claim 1, wherein said BBBD comprises increased BBB permeability, compared to a BBB control.

3. A method for determining a subject is at increased risk of developing a neurological disease or disorder, comprising determining a PSWE in said subject, wherein said PSWE has a median power frequency (MPF) of 3-10 Hz and is at least 5 seconds long, thereby determining said subject is at increased risk of developing the neurological disease or disorder.

4. The method of claim 1, wherein said PSWE having a MPF of 3-10 Hz and being at least 5 seconds long, is indicative of said subject being at increased risk of developing a neurological disease or disorder, compared to a control.

5. The method of claim 1, wherein said determining is by electroencephalogram (EEG).

6. The method of claim 1, wherein said PSWE is determined in the cerebral cortex of said subject.

7. The method of claim 3, wherein said neurological disease or disorder is selected from the group consisting of: Alzheimer's disease, Parkinson's disease, Havana syndrome, and a bipolar disorder.

8. The method of claim 3, wherein said subject is afflicted with a head trauma, BBB dysfunction (BBBD), or both.

9. The method of claim 1, further comprising a step of treating said subject with a BBB permeability-rectifying agent.

10. The method of claim 1, wherein said PSWE has a MPF of 5 Hz at most.

11. A method for treating a neurological disease or disorder in a subject in need thereof, comprising:
a. determining whether said subject has a PSWE having a MPF of 3-10 Hz and being at least 5 seconds long; and
b. administering to said subject determined as having a PSWE having a MPF of 3-10 Hz and being at least 5 seconds long, a therapeutically effective amount of a BBB permeability-rectifying agent,
thereby treating the neurological disease or disorder in the subject.

12. The method of claim 11, wherein said determining is by EEG.

13. The method of claim 11, wherein said PSWE is determined in the cerebral cortex of said subject.

14. The method of claim 11, wherein said neurological disease or disorder is selected from the group consisting of: Alzheimer's disease, Parkinson's disease, Havana syndrome, and a bipolar disorder.

15. The method of claim 11, wherein said subject is afflicted with a head trauma, BBBD, or both.

16. The method of claim 15, wherein said BBBD comprises increased BBB permeability, compared to a BBB control.

17. The method of claim 11, wherein said PSWE has a MPF of 5 Hz at most.

18. The method of claim 3, wherein said determining is by electroencephalogram (EEG).

19. The method of claim 3, wherein said PSWE is determined in the cerebral cortex of said subject.

20. The method of claim 3, further comprising a step of treating said subject with a BBB permeability-rectifying agent.
